(19) United States
(12) Patent Application Publication
(10) Pub. No.: US 2021/0115447 A1
(43) Pub. Date: Apr. 22, 2021

(54) NUCLEIC ACID APTAMERS TARGETING LYMPHOCYTE ACTIVATION GENE 3 (LAG-3) AND USES THEREOF

(71) Applicant: Oneness Biotech Co., Ltd., Taipei City (TW)

(72) Inventors: Yi-Chung CHANG, Taipei City (TW); Chien-Hao CHIANG, Taipei City (TW); Yi-Wei KAO, Taipei City (TW)

(73) Assignee: Oneness Biotech Co., Ltd., Taipei City (TW)

(21) Appl. No.: 17/251,736

(22) PCT Filed: Jun. 12, 2019
(86) PCT No.: PCT/CN2019/090832

§ 371 Date: Dec. 11, 2020

Related U.S. Application Data
(60) Provisional application No. 62/740,751, filed on Oct. 3, 2018.
(60) Provisional application No. 62/684,139, filed on Jun. 12, 2018.

(51) Int. Cl. C12N 15/115; A61P 35/00

(52) U.S. Cl. CPC C12N 15/115; C12N 2310/51; C12N 2310/16; A61P 35/00

(57) ABSTRACT

Nucleic acid aptamers capable of binding to lymphocyte activation gene 3 (LAG-3) and uses thereof for modulating immune responses. Such aptamers may comprise a G-rich motif, for example, GX₁GGGX₂GGTX₃A (SEQ ID No: 1), in which each of X₁ and X₂ are independently G, C, or absent, and X₃ is T or C, or L-(G)ₙ-L′, in which n is an integer of 5-9 inclusive, and L and L′ are nucleotide segments having complementary sequences. Also provided herein are multimeric nucleic acid aptamers containing a backbone moiety, which comprises a palindromic sequence.

### RELATED APPLICATIONS

This application claims the benefit of U.S. Provisional Application No. 62/684,139, filed on Jun. 12, 2018, and U.S. Provisional Application No. 62/740,751, filed Oct. 3, 2018 under 35 U.S.C. § 119, the entire content of each of which is incorporated by reference herein.

### BACKGROUND OF THE INVENTION

Activated T cells express multiple co-inhibitory molecules, known as immune checkpoint molecules, to modulate T cell responses. Exemplary immune checkpoint molecules include programmed cell death protein 1 (PD-1), lymphocyte-activated gene 3 (LAG-3), and cytotoxic T-lymphocyte-associated protein 4 (CTLA-4). These immune checkpoint molecules play important roles in maintaining immune homeostasis and preventing autoimmunity.

Immune checkpoint molecules are often activated in cancer, leading to suppression of anti-tumor immune responses. Thus, immune checkpoint inhibitor therapies provide effective long-term treatment for a variety of cancers. However, only a subset of cancer patients was found to respond to these treatments. It is therefore of great interest to develop new agents for inhibiting immune checkpoint targets for use in the treatment of cancer and other diseases via modulating immune cell activity, such as T cell proliferation and activation.

### SUMMARY OF THE INVENTION

The present disclosure is based, at least in part, on the development of nucleic acid aptamers, in monomeric form or in tetramer form, that bind human lymphocyte activation gene 3 (LAG-3) with high affinity and modulate immune responses via, e.g., disrupting the interaction between LAG-3 and MHC class II molecules. Surprisingly, such nucleic acid aptamers exhibited anti-tumor activity alone and enhanced the anti-tumor activity of checkpoint inhibitors such as anti-PD-1 antibodies.

Accordingly, one aspect of the present disclosure features a nucleic acid aptamer capable of binding to human LAG-3. Such a nucleic acid aptamer may comprise a nucleotide motif of: GX₁GGGX₂GGTX₃A (SEQ ID NO:1), in which each of X₁ and X₂ are independently G, C, or absent, and X₃ is T or C. In one example, the nucleotide motif can be GGGGGGGGTTA (SEQ ID NO:2). Alternatively, the nucleic acid aptamer may comprise a nucleotide motif of: L-(G)ₙ-L′, in which n is an integer of 5-9 inclusive (SEQ ID NOs:3-7), and L and L′ are nucleotide segments having complementary sequences (e.g., each containing 5-8 nucleotides).

Any of the nucleic acid aptamers described herein may comprise a nucleotide sequence at least 85% (e.g., at least 90%, at least 95%, or above) identical to one of the following nucleotide sequences:

(SEQ ID NO: 8)

| | |
|---|---|
| (i) | TGGGGGGGGTTAGTTCAATACATGCGGGCG; |

(SEQ ID NO: 9)

| | |
|---|---|
| (ii) | TGGGGGGGGGTTAGACTTACACTCTTATTCG; |

(SEQ ID NO: 10)

| | |
|---|---|
| (iii) | AGAGGGGGGGGTTAGCTGCTTTAACTCATG; |
| and | |

(SEQ ID NO: 11)

| | |
|---|---|
| (iv) | AGGGGGGGGGTTACTGCGCATGTATCTCAG. |

In some examples, the nucleic acid aptamer comprises one of the above-noted nucleotide sequences.

In one example, the nucleic acid aptamer comprises the nucleotide sequence of TGGGGGGGGTTAGTTCAATACATG (SEQ ID NO:12). Specific examples include:

(SEQ ID NO: 13)

| | |
|---|---|
| (a) | TCCCTACGGCGCTAACTGGGGGGGGTTAGTTCAATACATGCGGGCG |
| | GCCACCGTGCTACAAC; |

(SEQ ID NO: 14)

| | |
|---|---|
| (b) | ACGGCGCTAACTGGGGGGGGTTAGTTCAATACATG; |

(SEQ ID NO: 15)

| | |
|---|---|
| (c) | GCTAACTGGGGGGGGTTAGTTCAATACATGCGGGC; |
| and | |

(SEQ ID NO: 16)

| | |
|---|---|
| (d) | CTGGGGGGGGTTAGTTCAATACATGCGGGCGGCCA. |

In another aspect, the present disclosure features a multimeric nucleic acid aptamer (e.g., a tetramer aptamer), comprising a first polynucleic acid, a first nucleic acid aptamer, and a second nucleic acid aptamer. The first polynucleic acid may comprise a nucleotide sequence formula of 5′-X-L₁-Y-L₂-Z-3′, in which each of X and Z is a nucleotide segment complementary to a portion of the first nucleic acid aptamer and/or the second nucleic acid aptamer, each of L₁ and L₂ independently is a linker, and Y is a nucleotide segment having a palindromic sequence. The first nucleic acid aptamer and the second nucleic acid aptamer can form duplexes with the X and Z regions of the first polynucleic acid.

In some embodiments, the multimeric nucleic acid aptamer described herein may further comprise a second polynucleic acid, a third nucleic acid aptamer, and a fourth nucleic acid aptamer. The second polynucleic acid comprises a nucleotide sequence formula of 5′-X′-L₁′-Y-L₂′-Z′-3′, in which each of X′ and Z′ is a nucleotide segment complementary to a portion of the third nucleic acid aptamer and/or the fourth nucleic acid aptamer, each of L₁′ and L₂′ independently is a linker or is absent, and Y is the nucleotide segment having the palindromic sequence. The third nucleic acid aptamer and the fourth nucleic acid aptamer can form duplexes with the X′ and Z′ regions of the second polynucleic acid. The first polynucleic acid and the second polynucleic acid can form a duplex at the palindromic sequence in region Y.

In some embodiments, the multimeric nucleic acid aptamer described herein may comprise at least two nucleic acid aptamers specific to a same target molecule of interest. In some examples, the multimeric nucleic acid aptamer described herein may comprise at least two identical nucleic acid aptamers. In some examples, all of the nucleic acid aptamer moieties in the multimeric nucleic acid aptamer are identical.

In other embodiments, the multimeric nucleic acid aptamer described herein may comprise at least two nucleic acid aptamers specific to different target molecules of interest. In some examples, the multimeric nucleic acid aptamer described herein may comprise at least two different nucleic acid aptamers. In some examples, all of the nucleic acid aptamer moieties in the multimeric nucleic acid aptamer are different.

In some embodiments, at least one of the first nucleic acid aptamer, the second nucleic acid aptamer, the third nucleic acid aptamer, and the fourth nucleic acid aptamer in any of the multimeric nucleic acid aptamers can be those nucleic acid aptamers disclosed herein that are capable of binding to human LAG-3.

In yet another aspect, the present disclosure provides a multimeric nucleic acid complex, comprising a first polynucleic acid and optionally a second polynucleic acid. The first polynucleic acid comprises a nucleotide sequence formula of 5′-X-L₁-Y-L₂-Z-3′, in which X represents a first nucleic acid, Z represents a second nucleic acid, each of L₁ and L₂ independently is a linker, and Y is a nucleotide segment having a palindromic sequence. The second polynucleic acid comprises a nucleotide sequence formula of 5′-X′-L₁′-Y′-L₂′-Z′-3′, in which X′ represents a third nucleic acid, Z′ represents a fourth nucleic acid, each of L₁′ and L₂′ independently is a linker or is absent, and Y is the nucleotide segment having the palindromic sequence; wherein the first polynucleic acid and the second polynucleic acid form a duplex at the palindromic sequence region.

In some embodiments, the first nucleic acid, the second nucleic acid, the third nucleic acid, the fourth nucleic acid, or a combination thereof in the multimeric nucleic acid complex can be nucleic acid aptamers (identical or different). In other embodiments, the first nucleic acid, the second nucleic acid, the third nucleic acid, the fourth nucleic acid, or a combination thereof can be antisense oligonucleotides or siRNAs (identical or different). In yet other embodiments, at least one of the first nucleic acid, the second nucleic acid, the third nucleic acid, and the fourth nucleic acid is conjugated to a therapeutic agent, for example, a small molecule drug, a peptide drug, or a protein drug.

In some embodiments, at least two of the first nucleic acid, the second nucleic acid, the third nucleic acid and the fourth nucleic acid are specific to a same target molecule of interest. For example, at least two of the first nucleic acid, the second nucleic acid, the third nucleic acid and the fourth nucleic acid are identical nucleic acid aptamers. In some instances, all of the first nucleic acid, the second nucleic acid, the third nucleic acid and the fourth nucleic acid are identical nucleic acid aptamers.

In some embodiments, at least two of the first nucleic acid, the second nucleic acid, the third nucleic acid and the fourth nucleic acid are specific to different target molecules of interest. For example, at least two of the first nucleic acid, the second nucleic acid, the third nucleic acid and the fourth nucleic acid are different nucleic acid aptamers. In some instances, all of the first nucleic acid, the second nucleic acid, the third nucleic acid and the fourth nucleic acid are different nucleic acid aptamers. In some examples, at least one of the first nucleic acid, the second nucleic acid, the third nucleic acid, and the fourth nucleic acid is an anti-LAG3 nucleic acid aptamer disclosed herein.

In some examples, at least one of the first nucleic acid, the second nucleic acid, the third nucleic acid, and the fourth nucleic acid is a nucleic acid aptamer, and at least one of the other nucleic acids is an antisense oligonucleotide, a siRNA or conjugated to the therapeutic agent.

In some embodiments, the multimeric nucleic acid complex disclosed herein may further comprise a nucleic acid set, which comprises a fifth nucleic acid, a six nucleic acid, a seventh nucleic acid, an eighth nucleic acid, or a combination thereof, wherein each nucleic acid of the nucleic acid set comprises a portion that is complementary to the first nucleic acid, the second nucleic acid, the third nucleic acid, or the fourth nucleic acid and forms duplex with the first nucleic acid, the second nucleic acid, the third nucleic acid, or the fourth nucleic acid. The fifth nucleic acid, the sixth nucleic acid, the seventh nucleic acid, the eighth nucleic acid, or a combination thereof may comprise a nucleic acid aptamer, an antisense oligonucleotide, or a siRNA. Alternatively, at least one of the fifth nucleic acid, the sixth nucleic acid, the seventh nucleic acid, and the eighth nucleic acid is conjugated to a therapeutic agent, e.g., a small molecule drug, a peptide drug, or a protein drug.

In some instances, at least two of the fifth nucleic acid, the sixth nucleic acid, the seventh nucleic acid and the eighth nucleic acid are specific to a same target molecule of interest. For example, at least two (e.g., all) of the fifth nucleic acid, the sixth nucleic acid, the seventh nucleic acid and the eighth nucleic acid comprise an identical nucleic acid aptamer. In other instances, at least two (e.g., all) of the fifth nucleic acid, the sixth nucleic acid, the seventh nucleic acid and the eighth nucleic acid are specific to different target molecules of interest. In some examples, at least one of the fifth nucleic acid, the sixth nucleic acid, the seventh nucleic acid, and the eighth nucleic acid is an anti-LAG3 nucleic acid aptamer disclosed herein.

In some embodiments, at least one of the fifth nucleic acid, the sixth nucleic acid, the seventh nucleic acid, and the eighth nucleic acid comprises a nucleic acid aptamer, and at least one of the other nucleic acids comprises an antisense oligonucleotide, an siRNA or is conjugated to the therapeutic agent.

In any of the multimeric nucleic acid complexes (e.g., aptamers) described herein one or more of L₁, L₂, L₁′, and L₂′ (if applicable) are linkers such as polyA or polyT segments, which may consists of 4-10 A or T nucleotides. Alternatively or in addition, the palindromic sequence consists of 8, 10, 12, 14, or 16 nucleotides. In some examples, the palindromic sequence is (A/T)₄(C/G)₄(A/T)₄. In some embodiments, any one of the X and X′, L₁ and L₁′, L₂ and L₂′, and the Z and Z′ (if applicable) in the first polynucleic acid and the second polynucleic acid in the multimeric nucleic acid aptamers described herein are identical.

In addition, the present disclosure features a method for modulating immune responses, comprising administering to a subject in need thereof a pharmaceutical composition that comprises any of the nucleic acid aptamers binding to LAG-3 (in monomeric form or in multimeric form as described herein) and a pharmaceutically acceptable carrier. Such a pharmaceutical composition, which may be formulated for intravenous injection, is also within the scope of the present disclosure. In some embodiments, any of the anti-LAG3 aptamers described herein is administered to a subject by only a single dose.

In some embodiments, the subject may be a human patient having, suspected of having, or at risk for a cancer. Examples include, but are not limited to, lung cancer, melanoma, colorectal cancer, renal-cell cancer, urothelial carcinoma, and Hodgkin's lymphoma. In some examples, the pharmaceutical composition may be given to the patient in an amount sufficient to enhance T cell activity and/or inhibit cancer growth.

Moreover, the present disclosure features a method for detecting presence of LAG-3-positive cells, comprising: (i) contacting cells suspected of expressing LAG-3 with any of the nucleic acid aptamers described herein that bind LAG-3 (in monomeric form or in multimeric form), wherein nucleic acid aptamer is conjugated to a detection agent; and (ii) measuring a signal released from the detection agent conjugated to the nucleic acid aptamer that is bound to a cell; wherein intensity of the signal is indicative of presence or level of LAG-3-positive cells. In some embodiments, the contacting step (i) is performed by administering the nucleic acid aptamer or multimeric nucleic acid aptamer to a subject in need thereof.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIGS. 1A-1E are diagrams showing binding activity of candidate LAG-3 aptamer candidates. 1A: a graph showing results of a LAG-3/MHC-II bioassay using LAG-3 aptamer candidates as indicated. 1B: a schematic diagram of a conserved motif (SEQ ID NO:1) identified in exemplary LAG-3 aptamers. 1C: a graph showing results of in vitro binding of LAG-3 aptamers to His-tagged recombinant LAG-3 attached to nickel coated beads. 1D: a graph showing results of in vitro binding of LAG-3 aptamers to His-tagged recombinant LAG-3 attached to nickel coated wells of a plate. 1E: is a graph showing results of in vitro binding of truncated versions of LAG-3 aptamer B4 to His-tagged recombinant LAG-3 attached to nickel coated beads.

FIGS. 2A-2E are graphs showing molar ratios of backbone molecules and LAG-3 aptamer B4_FL in LAG-3 tetramers as determined using size exclusion chromatography. 2A: 1:1:10 molar ratio of backbone:backbone:aptamer. 2B: 1:1:8 molar ratio of backbone:backbone:aptamer. 2C: 1:1:6 molar ratio of backbone:backbone:aptamer. 2D: 1:1:5 molar ratio of backbone:backbone:aptamer. 2E: 1:1:4 molar ratio of backbone:backbone:aptamer.

FIGS. 3A-3J are graphs showing molar ratios of backbone molecules and various LAG-3 aptamer sequences in LAG-3 tetramers as determined using size exclusion chromatography. 3A: molar ratio of backbone sequences R1, R2 and aptamer sequence B4_FL in LAG-3 aptamer tetramers thus formed. 3B: molar ratio of backbone sequences R1_B10_P16 and R2_B10_P16 and aptamer sequence B4_SL3_P16 in LAG-3 aptamer tetramers thus formed. 3C: molar ratio of backbone sequences R1_B10_P16 and R2_B10_P16 and aptamer sequence B4_SL8_P16 in LAG-3 aptamer tetramers thus formed. 3D: molar ratio of backbone sequences R1_B10_P16 and R2_B10_P16 and aptamer sequence B4_SL11_P16 in LAG-3 aptamer tetramers thus formed. 3E: molar ratio of backbone sequences R1_B10_P10 and R2_B10_P10 and aptamer sequence B4_SL3_P10 in LAG-3 aptamer tetramers thus formed. 3F: molar ratio of backbone sequences R1_B10_P10 and R2_B10_P10 and aptamer sequence B4_SL8_P10 in LAG-3 aptamer tetramers thus formed. 3G: molar ratio of backbone sequences R1_B10_P10 and R2_B10_P10 and aptamer sequence B4_SL11_P10 in LAG-3 aptamer tetramers thus formed. 3H: molar ratio of backbone sequences R1_B18_P10 and R2_B18_P10 and aptamer sequence B4_SL3_P10 in LAG-3 aptamer tetramers thus formed. 3I: molar ratio of backbone sequences R1_B18_P10 and R2_B18_P10 and aptamer sequence B4_SL8_P10 in LAG-3 aptamer tetramers thus formed. 3J: molar ratio of backbone sequences R1_B18_P10 and R2_B18_P10 and aptamer sequence B4_SL11_P10 in LAG-3 aptamer tetramers thus formed.

FIGS. 4A-4C are diagrams showing binding activity of candidate LAG-3 aptamer candidates. 4A: a graph showing results of a LAG-3/MHC-II bioassay using LAG-3 tetramers formed using sequences indicated in Table 4. 4B: a graph showing results of in vitro binding of LAG-3 tetramers to His-tagged recombinant LAG-3. 4C: a graph showing results of in vitro binding of LAG-3 tetramers to His-tagged recombinant LAG-3 from different species such as rat, mouse, and human.

FIG. 5 is a graph showing results of size exclusion chromatography of LAG-3 tetramers formed by aptamer sequenceB4-SL3.

FIGS. 6A-6C are graphs showing uses of LAG-3 aptamer tetramers for treating xenograft mice having colon tumor, which was induced by subcutaneous inoculation of CT26 colon carcinoma cells. 6A: an illustration of the treatment region for mice implanted with CT26 colon carcinoma cells. 6B: a graph showing tumor volume at various time points after mice were implanted with CT26 colon carcinoma cells. 6C: a picture of tumors extracted from mice at day 21 after implantation with CT26 colon carcinoma cells.

FIGS. 7A-7D are pictures showing agarose gel analysis of various backbone sequences having palindromic residues. 7A: a picture showing an agarose gel of backbone sequences having 8 palindromic residues. 7B: a picture showing an agarose gel of backbone sequences having 10 palindromic residues. 7C: a picture showing an agarose gel of backbone sequences having 12 palindromic residues. 7D: a picture showing an agarose gel of backbone sequences having 16 palindromic residues.

FIGS. 8A-8B are graphs showing results of size exclusion chromatography of LAG-3 tetramers formed using backbone sequences having palindromic residues. 8A: a graph showing results of size exclusion chromatography of LAG-3 tetramers formed using a backbone sequence having 12 palindromic residues. 8B: a graph showing results of size exclusion chromatography of LAG-3 tetramers formed using a backbone sequence having 8 palindromic residues.

FIG. 9 is a picture showing an agarose gel of backbone sequences having different 12 residue palindromic sequences. B12P16: SEQ ID NO:17. B12P16_1: SEQ ID NO:18. B12P16_2: SEQ ID NO:19. B12P16_3: SEQ ID NO:20. B12P16_4: SEQ ID NO:21. B12P16_5: SEQ ID NO:22. B12P16_6: SEQ ID NO:23.

FIG. 10A-10B include diagrams showing therapeutic effect of anti-LAG3 aptamer (tetramer) in combination with anti-PD-L1 antibody observed in a mouse model. FIG. 10A: a schematic illustration of exemplary dosing regimens. FIG. 10B: a diagram showing inhibition of tumor group in mice treated with the anti-LAG3 aptamer in combination with the anti-PD-L1 antibody.

FIG. 11 is a schematic illustration showing multiple exemplary multimeric nucleic acid complexes carrying aptamers, siRNAs, and/or therapeutic agents.

### DETAILED DESCRIPTION OF THE INVENTION

Lymphocyte-activation gene 3 (LAG-3), also known as CD223, is a cell surface molecule belonging to the immunoglobulin (Ig) superfamily. It is a type-I transmembrane cell surface protein with four extracellular Ig-like domains. LAG-3 is typically expressed on activated T cells, natural killer cells, B cells, and/or dendritic cells. The native ligand of LAG-3 is MHC Class II molecules, to which LAG-3 binds with higher affinity than CD4. As a checkpoint receptor, LAG-3 may negatively regulate T cell proliferation, activation, and homeostasis, similar to CTLA-4 and PD-1. LAG-3 may also playa role in maintaining the tolerogenic state of CD8+ cells and/or CD8+ cell exhaustion during chronic viral infection. Further, LAG-3 may also play a role in dendritic cell maturation and activation. In human, LAG-3 is encoded by the LAG3 gene. An exemplary amino acid sequence of human LAG-3 can be found under GenBank accession number NP_002277.4.

Provided herein are nucleic acid aptamers capable of binding to LAG-3 and blocking its interaction with MHC II, thereby modulating the immune responses mediated by the LAG-3/MHC II interaction. Exemplary anti-LAG-3 aptamers as described herein, either in monomeric form or in tetramer form, successfully inhibited tumor growth as observed in an animal model.

Accordingly, described herein are anti-LAG-3 aptamers (monomeric or multimeric), pharmaceutical compositions comprising such, and methods for enhancing immune activity and/or treating diseases such as cancer with the anti-LAG-3 aptamers disclosed herein. Also provided herein are designs of multimeric nucleic acid complexes, which can be used for delivering various therapeutic agents, including nucleic acid-based agents (e.g., aptamers, antisense oligonucleotides, and/or interfering RNAs such as siRNAs), protein-based agents (e.g., peptide drugs or protein drugs), or small molecule agents.

### Anti-LAG-3 Aptamers

Described herein are nucleic acid aptamers that bind to human LAG-3 and interfere with its interaction with MHC Class II molecules, which are the native ligand of LAG-3, thereby modulating immune responses, for example, those mediated by the interaction between LAG-3 and a MHC Class II molecule. Accordingly, the anti-LAG-3 aptamers disclosed herein would be effective in modulating immune responses, which may benefit treatment of certain diseases and disorders, such as cancer and immune-disorders (e.g., autoimmune disorders).

A nucleic acid aptamer as used herein refers to a nucleic acid molecule (DNA or RNA) having a binding activity for a particular target molecule (e.g., LAG-3). The aptamer can bind to a particular target molecule and thereby inhibit the activity of the target molecule, via, e.g., blocking the binding of the target molecule to a native ligand thereof, causing conformational changes of the target molecule, and/or blocking the activity center of the target molecule. The anti-LAG-3 aptamer of the present disclosure, in linear or circular form, may be a RNA, a DNA (e.g., a single-stranded DNA), a modified nucleic acid, or a mixture thereof. The anti-LAG-3 aptamers may be non-naturally molecules (e.g., containing a nucleotide sequence not existing in native genes or containing modified nucleotides not existing in nature). Alternatively or in addition, the anti-LAG-3 aptamers may not contain a nucleotide sequence that encodes a functional peptide. In some instances, the anti-LAG-3 aptamer may be monomeric, i.e., comprising one binding site for a target molecule. Alternatively, an anti-LAG3 aptamer can be multimeric, i.e., comprising 2 or more binding site for one or more target molecules. See below discussions.

The anti-LAG-3 nucleic acid aptamer disclosed herein may comprise a G-rich segment (e.g., which plays an important role for the binding to a LAG-3 molecule (e.g., a human LAG-3) and interfering its interaction with the MHC Class II ligand. In some embodiments, the anti-LAG-3 aptamer may comprise a nucleotide motif (a): GX₁GGGX₂GGTX₃A (SEQ ID NO:1), in which each of X₁ and X₂ can independently be G, C, or absent, and/or X₃ can be T or C. In some examples, X₃ can be T. Alternatively or in addition, X₁, X₂, or both can be absent. In other examples, X₁, X₂, or both can be G or C. In particular examples, X₁, X₂, or both are G. For example, the anti-LAG-3 aptamer may comprise the nucleotide motif of GGGGGGTTA (SEQ ID NO:24), GGGGGGGTTA (SEQ ID NO:25), GGGGGGGGTTA (SEQ ID NO:2), or GGGGGGGGGTTA (SEQ ID NO:26).

In other embodiments, the anti-LAG-3 aptamer may comprise a nucleotide motif (b): L-(G)ₙ-L′, in which n is an integer of 5-9, inclusive (e.g., 5, 6, 7, 8, or 9; SEQ ID Nos:3-7, respectively); L and L′ are nucleotide sequences having complementary sequences such that the anti-LAG-3 aptamer may have a hairpin structure with the L/L′ forming a stem region and the polyG segment forming whole or part of a loop structure. In some instances, a portion of segment L is complementary to the whole or a portion of the L′ segment. In other instances, a portion of segment L′ is complementary to the whole or a portion of the L segment.

Both motif (a) and motif (b) contain a polyG segment, which is expected to play an important role in binding of the aptamer to LAG-3. Accordingly, nucleic acid molecules comprising either motif are expected to be an anti-LAG-3 aptamer as described herein.

In some examples, the anti-LAG-3 aptamer disclosed herein may comprise a nucleotide sequence at least 85% (e.g., 90%, 95%, or 98%) identical to

| (SEQ ID NO: 8) | |
|---|---|
| (i) | TGGGGGGGGTTAGTTCAATACATGCGGGCG; |
| (SEQ ID NO: 9) | |
| (ii) | TGGGGGGGGGTTAGACTTACACTCTTATTCG; |
| (SEQ ID NO: 10) | |
| (iii) | AGAGGGGGGGGTTAGCTGCTTTAACTCATG; |
| or | |
| (SEQ ID NO: 11) | |
| (iv) | AGGGGGGGGGTTACTGCGCATGTATCTCAG. |

Such anti-PDL1 nucleic acid aptamer disclosed herein may comprise or consist of any of the nucleotide sequences (i)-(iv) above.

The “percent identity” of two nucleic acids is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In other embodiments, the anti-PDL1 aptamers described herein may contain up to 8 (e.g., up to 7, 6, 5, 4, 3, 2, or 1) nucleotide variations as compared with a reference sequence, such as anyone of nucleotide sequences (i)-(iv). Positions where such variations can be introduced can be determined based on, e.g., the secondary structures of the aptamers which may be predicted using a computer algorithm, such as Mfold. For example, a base pair in a double-strand stem region may be mutated to a different base pair. Such mutations would maintain the base pair in the double-strand region at that position and thus would have no significant impact on the overall secondary structure of the aptamer. This type of mutations is well known to those skilled in the art. For example, an A-T pair may be mutated to a T-A pair. Alternatively, it may be mutated to a G-C or a C-G pair. In another example, a G-C pair may be mutated to a C-G pair. Alternatively, it may be mutated to an A-T pair or a T-A pair. Preferably, the one or more variations are at positions outside the core sequence GGGGGGTTAA (SEQ ID NO:27), GGGGGGGTTA (SEQ ID NO:28), or GGGGGGGGTTA (SEQ ID NO:29).

In some examples, the anti-LAG-3 aptamer comprises the nucleotide sequence of TGGGGGGGGTTAGTTCAATACATG (SEQ ID NO:12). Examples include, but are not limited to:

(SEQ ID NO: 13)
TCCCTACGGCGCTAACTGGGGGGGGTTAGTTCAATACATGCGGGCGGCCA
CCGTGCTACAAC;
(SEQ ID NO: 14)
ACGGCGCTAACTGGGGGGGGTTAGTTCAATACATG;
(SEQ ID NO: 15)
GCTAACTGGGGGGGGTTAGTTCAATACATGCGGGC;
and
(SEQ ID NO: 16)
CTGGGGGGGGTTAGTTCAATACATGCGGGCGGCCA.

Any of the anti-LAG-3 aptamers may further contain an anchor segment at the 5′ end, the 3′ end, or both. When an aptamer contains anchor segments at both the 5′ end and the 3′ end, the two anchor segments may be identical or different. The anchor segment may serve as a primer binding site, which can be used for amplifying the aptamer sequences. Alternatively or in addition, the anchor segment may serve as a binding site for attaching the aptamer to a backbone nucleic acid via base pairing to form multimeric anti-LAG-3 aptamers. See discussions below. Exemplary anchor sequences include 5′-TCCCTACGGCGCTAAC-3′ (SEQ ID NO:30) and 5′-GCCACCGTGCTACAAC-3′ (SEQ ID NO:31). An anti-LAG-3 aptamer as described herein may contain the whole anchor sequence noted above or a portion thereof. Exemplary aptamers containing anchor sequences are provided below (anchor sequence italicized; core sequence in boldface):

(SEQ ID NO: 13)
5′-TCCCTACGGCGCTAACTGGGGGGGGTTAGTTCAATACATGCGGGCGG
CCACCGTGCTACAAC-3′
(SEQ ID NO: 32)
5′-TCCCTACGGCGCTAACTGGGGGGGGGTTAGACTTACACTCTTATTCG
GCCACCGTGCTACAAC-3′
(SEQ ID NO: 33)
5′-TCCCTACGGCGCTAACAGAGGGGGGGGTTAGCTGCTTTAACTCATGG
CCACCGTGCTACAAC-3′
(SEQ ID NO: 34)
5′-TCCCTACGGCGCTAACAGGGGGGGGGTTACTGCGCATGTATCTCAGG
CCACCGTGCTACAAC-3′

Any of the anti-PDL1 aptamers disclosed herein may contain about 30-100 nucleotides (nts) in length (e.g., 35-100 nts). In some embodiments, the nucleic acid aptamer comprising a nucleic acid motif is about 40-80 nts, 40-65 nts, 40-62 nts, 50-80 nts, 60-80 nts, or 70-80 nts. In some embodiments, the nucleic acid aptamer comprising a nucleic acid motif is about 30-70 nts, 30-65 nts, 30-62 nts, 30-60 nts, 30-50 nts, or 30-40 nts. In some specific examples, the length of the anti-LAG-3 aptamers may range from about 50 nts to about 60 nts.

In general, the terms “about” and “approximately” mean within an acceptable error range for the particular value as determined by one of ordinary skill in the art. “About” can mean a range of less than 30%, preferably less than 20%, more preferably less than 10%, more preferably less than 5%, and more preferably still less than 1% of a given value.

In some embodiments, the anti-LAG-3 aptamers described herein may bind to an LAG-3 (e.g., human LAG-3) with a dissociation constant (Kd) lower than 20 nM (e.g., 15 nM, 10 nM, 5 nm, 1 nm, or less). The anti-LAG-3 aptamer may specifically bind human LAG-3. Alternatively, the aptamer may bind to LAG-3 molecules from different species (e.g., human, mouse, or rat). When binding to a LAG-3 molecule expressed on the cell surface, such an aptamer may inhibit the activity of LAG-3 (thus increasing immune cell activity such as T cell activity) by at least 20% (e.g., 40%, 50%, 80%, 100%, 2-fold, 5-fold, 10-fold, 100-fold, or 1,000-fold). The inhibitory activity of an anti-LAG-3 aptamer on LAG-3 (and thus the activation in enhancing immune cell activity such as T cell activity) may be determined by methods known in the art, e.g., T cell proliferation assays, which have been described previously, such as in Clay T. M., et al., Assays for Monitoring Cellular Immune Responses to Active Immunotherapy of Cancer, Clin Cancer Res., May 2001, 7, 1127; the relevant teachings of which are incorporated by reference herein. It should be appreciated that the methods for measuring T cell activity provided herein are exemplary and are not meant to be limiting.

In some embodiments, any of the anti-LAG-3 aptamers described herein may contain non-naturally-occurring nucleobases, sugars, or covalent internucleoside linkages (backbones). Such a modified oligonucleotide confers desirable properties, for example, enhanced cellular uptake, improved affinity to the target nucleic acid, and increased in vivo stability.

In one example, the aptamer described herein has a modified backbone, including those that retain a phosphorus atom (see, e.g., U.S. Pat. Nos. 3,687,808; 4,469,863; 5,321,131; 5,399,676; and 5,625,050) and those that do not have a phosphorus atom (see, e.g., U.S. Pat. Nos. 5,034,506; 5,166,315; and 5,792,608). Examples of phosphorus-containing modified backbones include, but are not limited to, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having 3′-5′ linkages, or 2′-5′ linkages. Such backbones also include those having inverted polarity, i.e., 3′ to 3′, 5′ to 5′ or 2′ to 2′ linkage. Modified backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In another example, the aptamers described herein include one or more substituted sugar moieties. Such substituted sugar moieties can include one of the following groups at their 2′ position: OH; F; O-alkyl, S-alkyl, N-alkyl, O-alkenyl, S-alkenyl, N-alkenyl; O-alkynyl, S-alkynyl, N-alkynyl, and O-alkyl-O-alkyl. In these groups, the alkyl, alkenyl and alkynyl can be substituted or unsubstituted C1 to C10 alkyl or C2 to C10 alkenyl and alkynyl. They may also include at their 2′ position heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide. Preferred substituted sugar moieties include those having 2′-methoxyethoxy, 2′-dimethylaminooxyethoxy, and 2′-dimethylaminoethoxyethoxy. See Martin et al., Helv. Chim. Acta, 1995, 78, 486-504.

Alternatively or in addition, aptamers described herein include one or more modified native nucleobases (i.e., adenine, guanine, thymine, cytosine and uracil). Modified nucleobases include those described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition, 1991, 30, 613, and Sanghvi, Y S., Chapter 15, Antisense Research and Applications, pages 289-302, CRC Press, 1993. Certain of these nucleobases are particularly useful for increasing the binding affinity of aptamer molecules to their targeting sites. These include 5-substituted pyrimidines, 6-azapyrimidines and N-2, N-6 and 0-6 substituted purines (e.g., 2-aminopropyl-adenine, 5-propynyluracil and 5-propynylcytosine). See Sanghvi, et al., eds., Antisense Research and Applications, CRC Press, Boca Raton, 1993, pp. 276-278).

Alternatively or in addition, the anti-PDL1 aptamers as described herein may comprise one or more locked nucleic acids (LNAs). An LNA, often referred to as inaccessible RNA, is a modified RNA nucleotide, in which the ribose moiety is modified with an extra bridge connecting the 2′ oxygen and 4′ carbon. This bridge “locks” the ribose in the 3′-endo (North) conformation, which is often found in the A-form duplexes. LNA nucleotides can be used in any of the anti-PDL1 aptamers described herein. In some examples, up to 50% (e.g., 40%, 30%, 20%, or 10%) of the nucleotides in an anti-PDL1 aptamer are LNAs. In some examples, an anti-PDL1 aptamer may comprise 10, 8, 6, 5, 4, 3, 2, or 1 LNA.

Any of the aptamers described herein can be prepared by conventional methods, e.g., chemical synthesis or in vitro transcription. Their intended bioactivity as described herein can be verified by, e.g., those described in the Examples below. Vectors for expressing any of the anti-PDL1 aptamers are also within the scope of the present disclosure.

Any of the aptamers described herein may be conjugated to one or more polyether moieties, such as polyethylene glycol (PEG) moieties, via covalent linkage, non-covalent linkage, or both. Accordingly, in some embodiments, aptamers described herein are PEGylated. The disclosure is not meant to be limiting with respect to a PEG moiety of a specific molecular weight. In some embodiments, the polyethylene glycol moiety has a molecular weight ranging from 5 kDa to 100 kDa, 10 kDa to 80 kDa, 20 kDa to 70 kDa, 20 kDa to 60 kDa, 20 kDa to 50 kDa, or 30 kDa to 50 kDa. In some examples, the PEG moiety has a molecular weight of 40 kDa. The PEG moiety conjugated to the anti-PDL1 aptamer described herein can be linear or branched. It may be conjugated to the 5′ end of the nucleic acid aptamer, the 3′ end of the aptamer, or both. When needed, the PEG moiety can be conjugated to the 3′ end of the nucleic acid aptamer covalently.

Methods for conjugating PEG moieties to nucleic acids are known in the art and have been described previously, for example, in PCT Publication No. WO 2009/073820, the relevant teachings of which are incorporated by reference herein. It should be appreciated that the PEG conjugated nucleic acid aptamers and methods for conjugating PEG to the nucleic acid aptamers described herein, are exemplary and not meant to be limiting.

### Multimeric Nucleic Acid Aptamers

The present disclose also provides nucleic acid aptamers in multimeric format, i.e., containing more than one aptamer binding moieties for binding to the same or different target molecules of interest. In some instances, the multimeric aptamer is a tetramer containing four aptamer binding moieties, which may be specific to the same target molecule or different target molecules. A multimeric aptamer would be expected to exhibit higher binding activity to a target molecule when it contains multiple binding moieties to the same target molecule as relative to the same aptamer binding moiety in monomeric format. Further, a multimeric aptamer would possess multiple binding specificities when containing multiple binding moieties to different target molecules, allowing for binding and modulating multiple targets simultaneously.

The multimeric nucleic acid aptamers described herein may comprise a backbone moiety, which may be conjugated to multiple aptamer moieties (e.g., 2, 3, or 4) covalently or via base-pairing. In some embodiments, the backbone moiety contains two nucleic acid molecules containing complementary sequences in the middle portion of each nucleic acid molecule. As used herein, complementary sequences, including completely or partially complementary sequences, refer to sequences capable of form a double-strand duplex via base-pairing according to the standard Watson-Crick complementary rules. The nucleic acid molecules of the backbone moiety further contain nucleotide sequences flanking the complementary sequence. Each of the flanking sequence may contain a docking site, which comprises a sequence complementary to a portion of an aptamer sequence (e.g., the anchor site discussed in the section above) such that the docking site can conjugate to the aptamer via base-pairing. Alternatively, the flanking sequence may comprise an aptamer moiety. The flanking sequences and the complementary sequence in each nucleic acid molecule of the backbone moiety may be covalently linked directly, or via a linker, such as a polyA or polyT segment.

In some embodiments, the backbone moiety may contain two identical nucleic acid molecules, which may contain a palindromic sequence as discussed below. A palindromic sequence, also known as an inverted-reverse sequence, refers to a nucleotide sequence (from 5′ to 3′ forward) that matches its complementary sequence reading from 5′ to 3′. Palindromic sequences tend to self-assemble to form stem-loop (hairpin) structures, which could be problematic in constructing multimeric aptamer complexes. Surprisingly, the present disclosure reports successful construction of tetramer aptamers using backbone moieties comprising palindromic sequences. Such tetramers demonstrated the desired bioactivities as shown in the Examples below.

In other embodiments, the backbone moiety may contain two different nucleic acid molecules. The backbone moiety may be conjugated to 2, 3, or 4 aptamer moieties. In some instances, all of the aptamer moieties are capable of binding to the same target molecule, e.g., identical aptamer moieties. In other instances, at least two aptamer moieties are capable of binding to different target molecules, i.e., multi-specific aptamers.

In some embodiments, the multimeric aptamer disclosed herein (e.g., a tetramer) may comprise a first polynucleic acid, a first nucleic acid aptamer, and a second nucleic acid aptamer, all of which form a complex. The first polynucleic acid comprises a nucleotide sequence formula of 5′-X-L₁-Y-L₂-Z-3′. Each of X and Z independently is a nucleotide segment containing a docking site that is complementary to a portion of the first nucleic acid aptamer and the second nucleic acid aptamer, respectively, such that the first and second nucleic acid aptamers can form base-pairs with the X and Z segments in the first polynucleic acid. In some instances, X and Z are identical. In other instances, X and Z are different (e.g., in length and/or sequences). L₁ and L₂ are each linkers, which may be a polyA or polyT segment (e.g., containing 4-10 A or T residues). In some examples, L₁ and L₂ are identical linkers. In other examples, L₁ and L₂ are different linkers (e.g., differ in sequence and/or length). In some instances, L₁, L₂, or both can be absent.

Y is a palindromic sequence, which may contain 8, 10, 12, 14, and 16 nucleotides. In some instances, the palindromic sequence contains the motif of (A/T)₄(G/C)₄(A/T)₄. Exemplary palindromic sequences include, but are not limited to, TCAGCTGA (SEQ ID NO:35), ATCAGCTGAT (SEQ ID NO:36), ATATCGCGATAT (SEQ ID NO:37), or ATATGACGCGTCATAT (SEQ ID NO:38).

The multimeric aptamer disclosed above may further comprise a second polynucleic acid, a third nucleic acid aptamer, and a fourth nucleic acid aptamer. The second polynucleic acid may comprise the nucleotide sequence formula of 5′-X′-L₁′-Y-L₂′-Z′-3′. Each of X′ and Z′ independently is a nucleotide segment containing a docking site that is complementary to a portion of the third nucleic acid aptamer and the fourth nucleic acid aptamer, respectively, such that the third and fourth nucleic acid aptamers can form base-pairs with the X′ and Z′ segments in the second polynucleic acid. In some instances, X′ and Z′ are identical. In other instances, X′ and Z′ are different (e.g., in length and/or sequences). In some examples, X and X′ and/or Z and Z′ are identical. In other examples, X differs from X′ and/or Z differs from Z′. L₁′ and L₂′ are each linkers, which may be a polyA or polyT segment (e.g., containing 4-10 A or T residues). In some examples, L₁′ and L₂′ are identical linkers. In other examples, L₁′ and L₂′ are different linkers (e.g., differ in sequence and/or length).

In some examples, the first polynucleotic acid and the second polynucleic acid in the multimeric aptamer described above are identical molecules. In other examples, the first polynucleotic acid and the second polynucleic acid differ in at least one aspect, for example, a different docking site and/or a different linker. In some instances, at least two aptamers (2, 3, or 4) in the multimeric aptamer complex bind to the same target molecule. In one example, the aptamers (2, 3, or 4) are identical aptamer molecules. In other instances, at least two aptamers (2, 3, or 4) in the multimeric aptamer complex bind to different target molecules.

Any of the multimeric aptamers described herein, for example, tetramers, may contain one or more of the anti-LAG-3 nucleic acid aptamers described herein. In some embodiments, the multimeric aptamer is a tetramer containing 1, 2, 3, or 4 anti-LAG-3 nucleic acid aptamers as described herein. When the tetramer contains 2 or more anti-LAG-3 aptamers, the anti-LAG-3 aptamer moieties may be identical, or different. In some examples, the tetramer contains four identical anti-LAG-3 moieties, which can be any of the anti-LAG-3 aptamers described herein.

The anti-LAG-3 aptamers may be conjugated to the backbone moiety in the multimeric aptamer via base-pairing. Alternatively, the anti-LAG-3 aptamers may linked covalently to the backbone nucleic acid to form a single polynucleotide chain. See above discussions.

### Multimeric Nucleic Acid Complexes

In addition, the present disclosure provides a universal design of multimeric (e.g., tetramer) nucleic acid molecules, using palindromic sequences. Palindromic sequences suitable for making multimeric nucleic acid molecules may contain 8, 10, 12, 14, or 16 nucleotides. In some instances, the palindromic sequence contains the motif of (A/T)₄(G/C)₄(A/T)₄. Exemplary palindromic sequences include, but are not limited to, TCAGCTGA (SEQ ID NO:35), ATCAGCTGAT (SEQ ID NO:36), ATATCGCGATAT (SEQ ID NO:37), or ATATGACGCGTCATAT (SEQ ID NO:38).

An exemplary tetramer nucleic acid complex may contain two polynucleic acid molecules, each comprising a palindromic sequence flanked by two nucleic acid segments. The nucleic acids of interest may be linked directly to the palindromic sequence or linked to the palindromic sequence via a linker, for example, those described herein. The two polynucleic acid molecules form a duplex via the palindromic sequence, thereby producing the multimeric nucleic acid complex as disclosed herein.

In some embodiments, at least one or all of the nucleic acid segments flanking the palindromic sequence in the two polynucleic acid molecules comprise nucleic acid-based therapeutic agents, such as nucleic acid aptamers (e.g., the anti-LAG3 nucleic acid aptamers disclosed herein), antisense oligonucleotides, and/or interfering RNAs such as siRNAs. Alternatively or in addition, at least one or all of the nucleic acid segments flanking the palindromic sequence in the two polynucleic acid molecules are conjugated to a therapeutic agent, which may be a peptide drug, a protein drug, or a small molecule drug. A peptide drug, a protein drug, or a small molecule drug refers to any peptide, protein, or small molecule that has a therapeutic activity.

In other embodiments, the multimeric nucleic acid complex disclosed herein may comprise one or more nucleic acids, each of which contain a segment that is complementary to a portion or all of one nucleic acid segment flanking the palindromic sequence in the two polynucleic acid molecules such that the additional nucleic acids form duplexes with the nucleic acid segment flanking the palindromic sequence. In some instances, one or more of the additional nucleic acids may comprise nucleic acid-based therapeutic agents (identical or different) such as nucleic acid aptamers, e.g., any of the anti-LAG3 aptamers disclosed herein, antisense oligonucleotides, and/or interfering RNAs such as siRNAs. In other instances, one or more of the additional nucleic acids may be conjugated to a non-nucleic acid based therapeutic agent, such as a peptide drug, a protein drug, or a small molecule drug.

In some instances, a multimeric nucleic acid complex disclosed herein may carry the same therapeutic agent as those described herein. In other instances, the multimeric nucleic acid complex disclosed herein may carry multiple therapeutic agents. In some embodiments, the multimeric nucleic acid complex may contain nucleic acids of interest of the same type (e.g., nucleic acid aptamers, antisense oligonucleotides, or siRNAs capable of binding to the same target). Alternatively, a multimeric nucleic acid complex described herein may contain nucleic acids of interest of different types (e.g., nucleic acid aptamers binding to different targets). In other embodiments, the multimeric nucleic acid complex disclosed herein may comprise multiple therapeutic agents, which may be different types of molecules (e.g., peptide, protein, nucleic acid, and/or small molecules). For example, the multimeric nucleic acid complex may comprise a nucleic acid aptamer targeting a biomarker associated with a disease and a therapeutic agent such that the aptamer could lead the therapeutic agent to locations where the biomarker presents to exert its therapeutic activity.

### Pharmaceutical Compositions

One or more of the anti-LAG-3 aptamers (in monomeric form or multimeric form as described herein, and/or in free form or PEG-conjugated form as also described herein) can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. See, e.g., Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

In some examples, the pharmaceutical composition described herein comprises liposomes containing any of the LAG-3-binding aptamers (in monomeric form or multimeric form, or a vector(s) for producing the aptamer), which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The anti-LAG-3 aptamers as described herein may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the LAG-3 binding aptamer, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic PDL1 binding aptamer compositions may be placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between and 20%. The fat emulsion can comprise fat droplets having a suitable size and can have a pH in the range of 5.5 to 8.0.

The emulsion compositions can be those prepared by mixing an anti-LAG-3 aptamer with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

### Therapeutic Applications

Any of the anti-LAG-3 aptamers, in monomeric form or multimeric form, in free form or PEG-conjugated form, all of which have been described herein, can be used to modulate immune activity, for example, promoting T cell proliferation, thereby effective in treating diseases or disorders that can benefit from modulation of immune responses, for example, cancer or immune disorders.

To practice the method disclosed herein, an effective amount of the pharmaceutical composition described herein that contains at least one anti-LAG-3 aptamer can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the anti-LAG-3 aptamer-containing composition as described herein can be aerosolized using a fluorocarbon formulation and a metered dose inhaler, or inhaled as a lyophilized and milled powder.

As used herein, “an effective amount” refers to the amount of each active agent required to confer therapeutic effect on the subject, either alone or in combination with one or more other active agents. In some embodiments, the therapeutic effect is reduced tumor burden, reduction of cancer cells, or increased immune activity. Determination of whether an amount of the LAG-3 binding aptamers achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. Frequency of administration may be determined and adjusted over the course of therapy, and is generally, but not necessarily, based on treatment and/or suppression and/or amelioration and/or delay of a target disease/disorder. Alternatively, sustained continuous release formulations of a LAG-3 binding aptamer may be appropriate. Various formulations and devices for achieving sustained release are known in the art.

In one example, dosages for an anti-LAG-3 aptamer as described herein may be determined empirically in individuals who have been given one or more administration(s) of the LAG-3 binding aptamer. Individuals are given incremental dosages of the antagonist. To assess efficacy of the antagonist, an indicator of the disease/disorder can be followed.

Generally, for administration of any of the anti-LAG-3 aptamers described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the LAG-3 binding aptamer, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the LAG-3 binding aptamer used) can vary over time. In one specific example, the LAG-3 binding aptamer as described herein may be given to a subject in need of the treatment (e.g., a human patient who needs modulation of immune responses) by a single dose.

In some embodiments, for an adult patient of normal weight, doses ranging from about 0.3 to 5.00 mg/kg may be administered. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of a LAG-3 binding aptamer as described herein will depend on the specific LAG-3 binding aptamer, the type and severity of the disease/disorder, whether the LAG-3 binding aptamer is administered for preventive or therapeutic purposes, previous therapy, the patient's clinical history and response to the antagonist, and the discretion of the attending physician. A clinician may administer a LAG-3 binding aptamer, until a dosage is reached that achieves the desired result. In some embodiments, the desired result is a decrease in tumor burden, a decrease in cancer cells, or increased immune activity. Methods of determining whether a dosage resulted in the desired result would be evident to one of skill in the art. Administration of one or more LAG-3 binding aptamers can be continuous or intermittent, depending, for example, upon the recipient's physiological condition, whether the purpose of the administration is therapeutic or prophylactic, and other factors known to skilled practitioners. The administration of a LAG-3 binding aptamer may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease, or reducing disease severity. Alleviating the disease does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

In some embodiments, the LAG-3 binding aptamers described herein are administered to a subject in need of the treatment at an amount sufficient to reduce tumor burden or cancer cell growth, by at least 5% (e.g., 10%, 20%, 30%, 40%, 50%, 60%, 70%, 80%, 90% or greater) in vivo. In other embodiments, the LAG-3 binding aptamers are administered in an amount effective in reducing the activity level of LAG-3 by at least 5% (e.g., 10%, 20%, 30%, 40%, 50%, 60%, 70%, 80%, 90% or greater). In other embodiments, the LAG-3 binding aptamers are administered in an amount effective in increasing immune activity by at least 5% (e.g., 10%, 20%, 30%, 40%, 50%, 60%, 70%, 80%, 90% or greater).

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularlly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble LAG-3 binding aptamers can be administered by the drip method, whereby a pharmaceutical formulation containing the LAG-3 binding aptamer and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the PDL1 binding aptamer, can be dissolved and administered in a pharmaceutical excipient such as Water-for-Injection, 0.9% saline, or 5% glucose solution.

In one embodiment, a LAG-3 binding aptamer is administered via site-specific or targeted local delivery techniques. Examples of site-specific or targeted local delivery techniques include various implantable depot sources of the LAG-3 binding aptamer or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., the LAG-3 binding aptamers described herein or vectors for producing such) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The subject to be treated by the methods described herein can be a mammal, such as a farm animals, sport animals, pets, primates, horses, dogs, cats, mice and rats. In one example, the subject is a human. The anti-LAG-3 aptamer-containing composition may be used for enhancing immune activity, for example, T cell activity, in a subject in need of the treatment. In some examples, the subject may be a human patient having, suspected of having, or at risk for a cancer, such as lung cancer, melanoma, colorectal cancer, renal-cell cancer, urothelial carcinoma, or Hodgkin's lymphoma. Such a patient can also be identified by routine medical practices.

A subject having a target disease or disorder (e.g., cancer or an immune disorder) can be identified by routine medical examination, e.g., laboratory tests, organ functional tests, CT scans, or ultrasounds. A subject suspected of having any of such target disease/disorder might show one or more symptoms of the disease/disorder. A subject at risk for the disease/disorder can be a subject having one or more of the risk factors associated with that disease/disorder. Such a subject can also be identified by routine medical practices.

The particular dosage regimen, i.e., dose, timing and repetition, used in the method described herein will depend on the particular subject (e.g., a human patient) and that subject's medical history.

In some embodiments, the anti-LAG-3 aptamer may be co-used with another suitable therapeutic agent (e.g., an anti-cancer agent an anti-viral agent, or an anti-bacterial agent). Alternatively or in addition, the anti-LAG-3 aptamer may also be used in conjunction with other agents that serve to enhance and/or complement the effectiveness of the agents.

Treatment efficacy for a target disease/disorder can be assessed by, e.g., a method described in the Examples below.

### Diagnostic Applications and Others

Any of the anti-LAG-3 aptamers can also be used for detecting presence of LAG-3 molecules and cells expressing such, or for delivering a therapeutic agent to LAG-3⁺ cells. The anti-LAG-3 aptamer may be chemically synthesized and be manipulated with functional groups for conjugation with a therapeutic agent or a detectable label (e.g., an imaging agent such as a contrast agent) for diagnostic purposes, either in vivo or in vitro. As used herein, “conjugated” or “attached” means two entities are associated, preferably with sufficient affinity that the therapeutic/diagnostic benefit of the association between the two entities is realized. The association between the two entities can be either direct or via a linker, such as a polymer linker. Conjugated or attached can include covalent or noncovalent bonding as well as other forms of association, such as entrapment, e.g., of one entity on or within the other, or of either or both entities on or within a third entity, such as a micelle.

In one example, an anti-LAG-3 aptamer as described herein can be attached to a detectable label, which is a compound that is capable of releasing a detectable signal, either directly or indirectly, such that the aptamer can be detected, measured, and/or qualified, in vitro or in vivo. Examples of such “detectable labels” are intended to include, but are not limited to, fluorescent labels, chemiluminescent labels, colorimetric labels, enzymatic markers, radioactive isotopes, and affinity tags such as biotin. Such labels can be conjugated to the aptamer, directly or indirectly, by conventional methods.

In some embodiments, the detectable label is an agent suitable for imaging a disease mediated by LAG-3/MHC II interaction, which can be a radioactive molecule, a radiopharmaceutical, or an iron oxide particle. Radioactive molecules suitable for in vivo imaging include, but are not limited to, ¹²²I, ¹²³I, ¹²⁴I, ¹²⁵I, ¹³¹I, ¹⁸F, ⁷⁵Br, ⁷⁶Br, ⁷⁶Br, ⁷⁷Br, ²¹¹At, ²²⁵Ac, ¹⁷⁷Lu, ¹⁵³Sm, ¹⁸⁶Re, ¹⁸⁸Re, ⁶⁷Cu, ²¹³Bi, ²¹²Bi, ²¹²Pb, and ⁶⁷Ga. Exemplary radiopharmaceuticals suitable for in vivo imaging include ¹¹¹In Oxyquinoline, ¹³¹I Sodium iodide, ^{99m}Tc Mebrofenin, and ^{99m}Tc Red Blood Cells, ¹²³I Sodium iodide, ^{99m}Tc Exametazime, ^{99m}Tc Macroaggregate Albumin, ^{99m}Tc Medronate, ^{99m}Tc Mertiatide, ^{99m}Tc Oxidronate, ^{99m}Tc Pentetate, ^{99m}Tc Pertechnetate, ^{99m}Tc Sestamibi, ^{99m}Tc Sulfur Colloid, ^{99m}Tc Tetrofosmin, Thallium-201, and Xenon-133. The reporting agent can also be a dye, e.g., a fluorophore, which is useful in detecting a disease mediated by LAG-3 in tissue samples.

In some embodiments, an anti-LAG-3 aptamer conjugated to a detectable label (e.g., an imaging agent) as disclosed herein is administered to a subject to assess LAG-3 levels in the subject. Such detection of LAG-3 may be used to identify relevant patients for anti-LAG-3 treatment (e.g., for treatment with an anti-LAG-3 pharmaceutical composition disclosed herein or for treatment with anti-LAG-3 antibody).

LAG-3 or LAG-3⁺ cells may be detected in a sample (e.g., a biological sample suspected of containing LAG-3, including but not limited to a blood sample and urine sample) in vitro using any of the aptamers described herein via a routine method. In some instances, the aptamer may be conjugated to a detectable label, which may release a signal, directly or indirectly, indicating the presence and/or level of LAG-3 in the sample. Alternatively, the anti-LAG-3 aptamer may be used for in vivo imaging of presence and localization of LAG-3 or LAG-3⁺ cells in a subject (e.g., a human patient as described herein). Results obtained from any of the diagnostic assays described herein (either in vitro or in vivo) may be indicative of a risk or state of a disease associated with LAG-3.

Diagnostic and Treatment Kits Containing anti-LAG-3 Aptamers

The present disclosure also provides kits for use in modulating (e.g., enhancing) immune activity (e.g., T cell activity), alleviating cancer (e.g., lung cancer, melanoma, colorectal cancer, or renal-cell cancer), and/or treating or reducing the risk for cancer. Such kits can include one or more containers comprising an aptamer that binds LAG-3, e.g., any of those described herein.

In some embodiments, the kit can comprise instructions for use in accordance with any of the methods described herein. For example, the included instructions can comprise a description of administration of the aptamer to treat, delay the onset, or alleviate a target disease as those described herein. The kit may further comprise a description of selecting an individual suitable for treatment based on identifying whether that individual has the target disease. In still other embodiments, the instructions comprise a description of administering the aptamer to an individual at risk of the target disease.

The instructions relating to the use of a LAG-3 binding aptamer generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating a disease or disorder associated with cancer, such as those described herein. Instructions may be provided for practicing any of the methods described herein.

In addition, the present disclosure provides kits for detecting or measuring the level of LAG-3 and/or LAG-3⁺ cells in a biological sample or for in vivo diagnostic purposes. Such a kit may comprise one or more of the anti-LAG-3 aptamers described herein, which may be conjugated to a detectable label as also described herein. The kit may further comprise one or more reagents for processing a biological sample and/or for producing or detecting a signal released from the detectable label, directly or indirectly. The kit may further include instructions for performing an assay for detecting or measuring the level of LAG-3 or LAG-3⁺ cells in a sample using the anti-LAG-3 aptamer included in the kit. The kit may comprise a description of how to process a biological sample and how to perform a suitable assay for measuring LAG-3 or LAG-3⁺ cells in the sample.

The kits as described herein are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is a LAG-3 binding aptamer as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present invention will employ, unless otherwise indicated, conventional techniques of molecular biology (including recombinant techniques), microbiology, cell biology, biochemistry and immunology, which are within the skill of the art. Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the systems and methods provided herein and are not to be construed in any way as limiting their scope.

### Example 1: Identification and Characterization of LAG-3 Aptamers

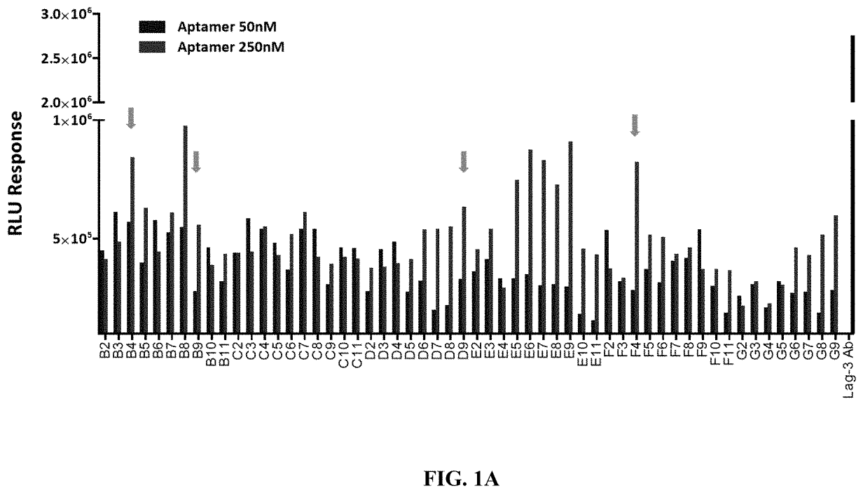

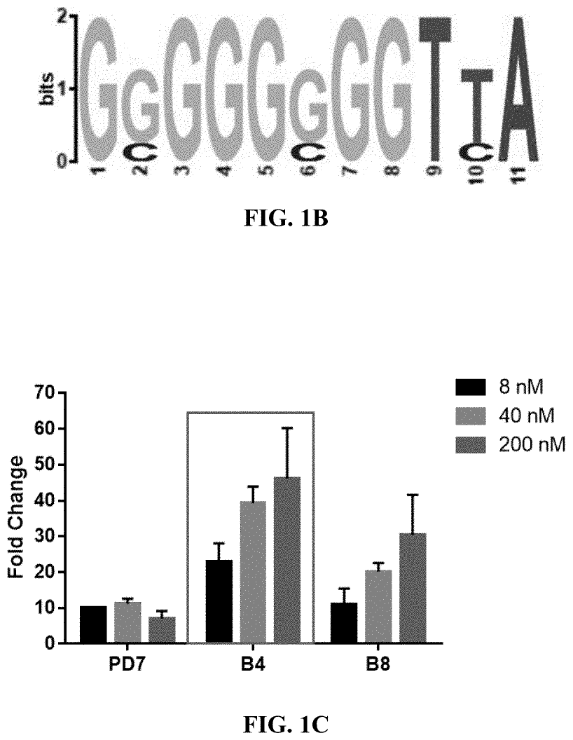

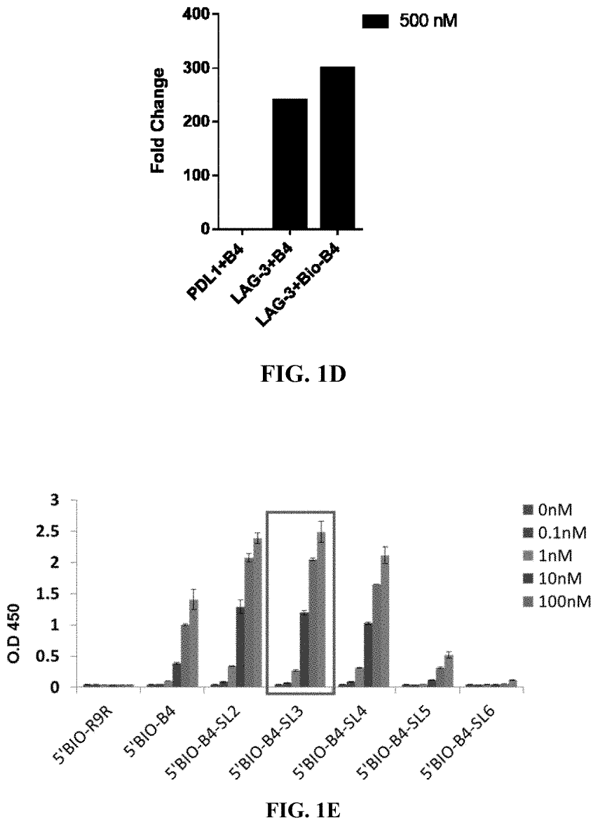

Candidate lymphocyte-activation gene 3 (LAG-3) aptamers were identified from a synthetic ssDNA library using a high-throughput SELEX assay with human recombinant LAG-3 as a target. Such LAG-3 aptamer candidates were subjected to next-generation sequencing, and tested for their activity in disruption of LAG-3 interaction with MHC-II using a LAG-3/MHC-II bioassay. Several LAG-3 aptamers were found to disrupt interaction between LAG-3 and MHC-II as indicated by expression of a luciferase reporter. Exemplary results are provided in FIG. 1A. Anti-LAG-3 antibody was used as a positive control. FIG. 1A.

LAG-3 aptamers B4, B8, D9 and F4 are marked with arrows in FIG. 1A, and sequences of these aptamers are provided in Table 1 below. Sequence alignment revealed a conserved motif in the identified LAG-3 aptamers as shown in FIG. 1B. Aptamers comprising this conserved motif are expected to bind to LAG-3 and disrupt its interaction with MHC-II.

To directly test binding of LAG-3 aptamers to LAG-3, in vitro binding assays were performed using His-tagged recombinant LAG-3 attached to nickel coated beads or nickel coated wells of a plate. Bound LAG-3 aptamers were eluted and detected by qPCR. Using the bead based assay, binding of aptamers B4 and B8 to recombinant LAG-3 increased in a concentration dependent manner. FIG. 1C. Using the plate based assay, aptamer B4 and a biotin tagged version of aptamer B4 (Bio-B4) were found to bind to recombinant LAG-3 in a similar manner. FIG. 1D.

TABLE 1
Sequences of Exemplary LAG-3 Aptamers.

| SEQ | |
|---|---|
| ID | Sequence 5′ → 3′ |
| 13 | TCCCTACGGCGCTAACTGGGGGGGGTTAGTTCAATACATGC |
| | GGGCGGCCACCGTGCTACAAC |
| | (B4) |
| 32 | TCCCTACGGCGCTAACTGGGGGGGGGTTAGACTTACACTCT |
| | TATTCGGCCACCGTGCTACAAC |
| | (B8) |
| 33 | TCCCTACGGCGCTAACAGAGGGGGGGGTTAGCTGCTTTAAC |
| | TCATGGCCACCGTGCTACAAC |
| | (D9) |
| 34 | TCCCTACGGCGCTAACAGGGGGGGGGTTACTGCGCATGTAT |
| | CTCAGGCCACCGTGCTACAAC |
| | (F4) |

* Conserved motif is shown in boldface and underlined.

To identify the minimal sequence of aptamer B4 that is involved in binding to LAG-3, various truncated versions of aptamer B4 were prepared and their binding activities to LAG-3 were investigated in the binding assays described herein, using R9R as a negative control. Aptamer sequences of these truncated versions of aptamer B4 and their dissociation constants (Kds) for binding to recombinant LAG-3 are shown in Table 2. Binding of aptamers B4, B4-SL2, B4-SL3, and B4-SL4 to recombinant LAG-3 increased in a concentration dependent manner. FIG. 1E. Truncated versions B4-SL5 and B4-SL6 showed minimal to no binding; these two truncated variants have a deletion of a portion of the conserved motif noted above. FIG. 1E.

TABLE 2
Truncated Aptamer B4 Sequences and Dissociation
Constants.

| | | Kd |
|---|---|---|
| Aptamer | Sequence | (nM) |
| 5′Bio-B4 | TCCCTACGGCGCTAACTGGGGGGGGTTAGTTCAAT | 38.14 |
| (SEQ ID | ACATGCGGGCGGCCACCGTGCTACAAC | |
| NO: 13) | | |
| 5′Bio-B4- | ACGGCGCTAACTGGGGGGGGTTAGTTCAATACATG | 8.05 |
| SL2 | | |
| (SEQ ID | | |
| NO: 14) | | |
| 5′Bio-B4- | GCTAACTGGGGGGGGTTAGTTCAATACATGCGGGC | 10.71 |
| SL3 | | |
| (SEQ ID | | |
| NO: 15) | | |
| 5′Bio-B4- | CTGGGGGGGGTTAGTTCAATACATGCGGGCGGCCA | 9.47 |
| SL4 | | |
| (SEQ ID | | |
| NO: 16) | | |
| 5′Bio-B4- | GGGGGTTAGTTCAATACATGCGGGCGGCCACCGTG | 91.35 |
| SL5 | | |
| (SEQ ID | | |
| NO: 39) | | |
| 5′Bio-B4- | TTAGTTCAATACATGCGGGCGGCCACCGTGCTACA | 123.6 |
| SL6 | | |
| (SEQ ID | | |
| NO: 40) | | |

* Conserved motif or portions thereof are shown in boldface and underlined.

These results demonstrated that the conserved motif shown in FIG. 1B is important for binding to recombinant LAG-3 and for disrupting LAG-3 interaction with MCH-II in cell cultures. Accordingly, aptamers comprising this conserved motif would be expected to possess the activity of binding to LAG-3 and disrupt its interaction with MCH-II.

### Example 2: Synthesis and Characterization of LAG-3 Aptamers in Tetramer Form

LAG-3 aptamers in tetramer form were constructed using two backbone sequences having complementary segments such that they can be annealed together via base pairing. Each of the backbone sequences can be attached to two aptamers at the 5′ and 3′ ends, thereby forming an aptamer tetramer. This attachment is achieved using a backbone sequence having a primer sequence at each end that is complementary to a primer sequence in an aptamer sequence which allows base pairing of an aptamer to each end of the backbone sequence. Exemplary aptamer sequences and backbone sequences are shown in Table 3.

TABLE 3
Exemplary Backbone and Aptamer Sequences.

| | Sequence |
|---|---|
| Aptamer | |
| B4_SL3_ | GCTAACTGGGGGGGGTTAGTTCAATACATG |
| P16 | CGGGCGCCACCGTGCTACAAC |
| | (SEQ ID NO: 41) |
| B4_SL11_ | GCTAACTGGGGGGGGTTAGTTCAATACAT |
| P16 | GGCCACCGTGCTACAAC |
| | (SEQ ID NO: 42) |
| B4_SL8_ | GCTAACTGGGGGGGGTTAGTTCAATGCCA |
| P16 | CCGTGCTACAAC |
| | (SEQ ID NO: 43) |
| B4_SL3_ | GCTAACTGGGGGGGGTTAGTTCAATACAT |
| P10 | GCGGGCGTGCTACAAC |
| | (SEQ ID NO: 44) |
| B4_SL11_ | GCTAACTGGGGGGGGTTAGTTCAATACAT |
| P10 | GGTGCTACAAC |
| | (SEQ ID NO: 45) |
| B4_SL8_ | GCTAACTGGGGGGGGTTAGTTCAATGTGC |
| P10 | TACAAC |
| | (SEQ ID NO: 46) |
| Backbone | |
| B18P16 | GTTGTAGCACGGTGGCTTTTTATTTAGGTG |
| | ACACTATAGTTTTTGTTGTAGCACGGTGGC |
| | (SEQ ID NO: 47) |
| B10P16 | GTTGTAGCACGGTGGCTTTTTAGGTGACAC |
| | TTTTTTGTTGTAGCACGGTGGC |
| | (SEQ ID NO: 48) |
| B10P10 | GTTGTAGCACTTTTTAGGTGACACTTTTTT |
| | GTTGTAGCAC |
| | (SEQ ID NO: 49) |
| B18P10 | GTTGTAGCACTTTTTATTTAGGTGACACTA |
| | TAGTTTTTGTTGTAGCAC |
| | (SEQ ID NO: 50) |

* Primer sequences are underlined.
P16 indicates a 16 residue primer, and P10 indicates a 10 residue primer.

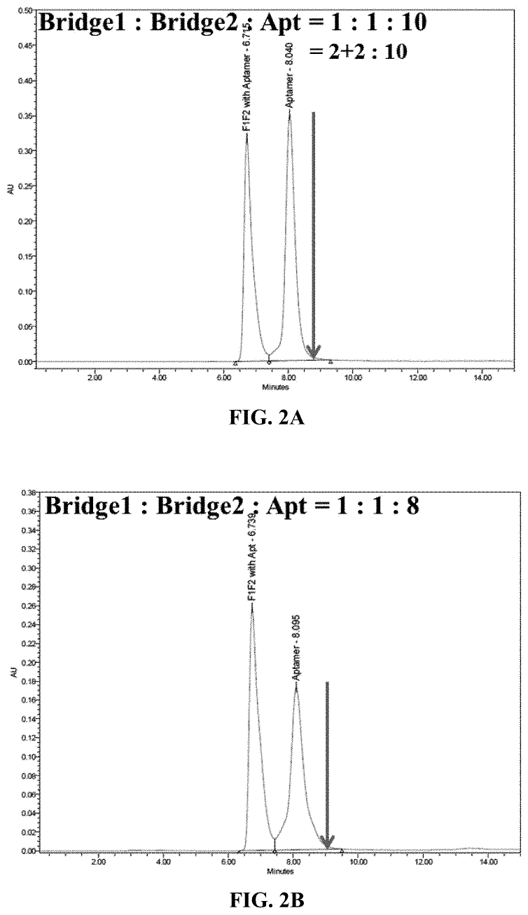

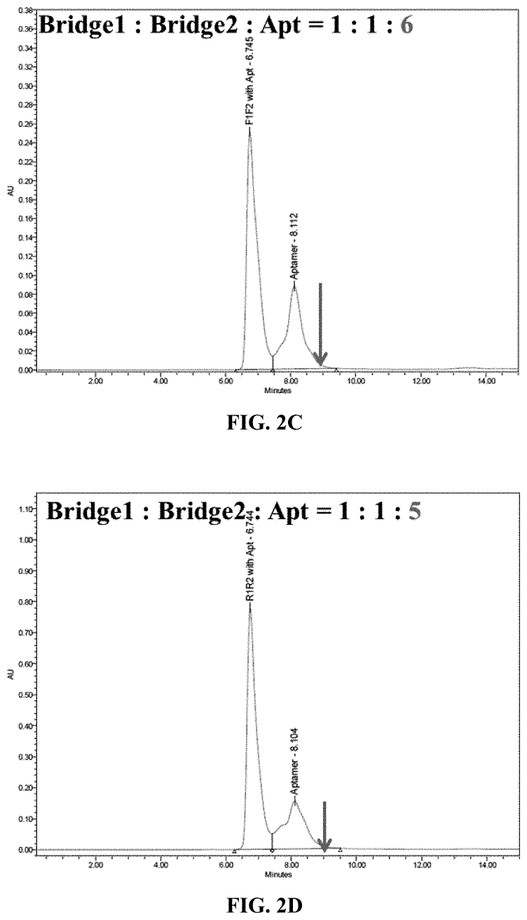

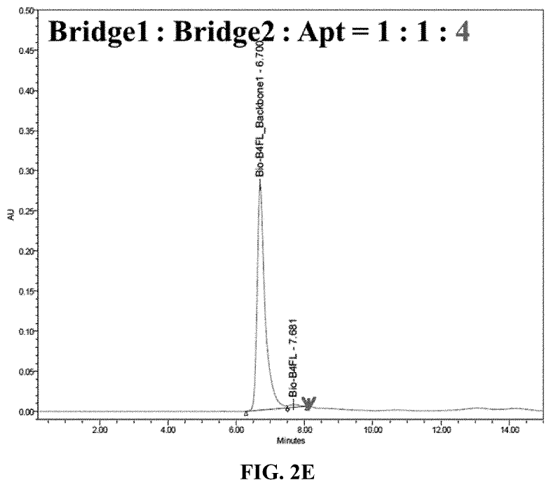

The backbone sequences and the aptamer sequences were mixed at various molar ratios and the tetramers thus formed were isolated by size exclusion chromatography. As shown in FIGS. 2A to 2E, the peak representing free aptamers decreased as the molar concentration of aptamers decreased. The resultant backbone/aptamer complexes are in tetramer form containing the two backbone molecules associated with 4 aptamers.

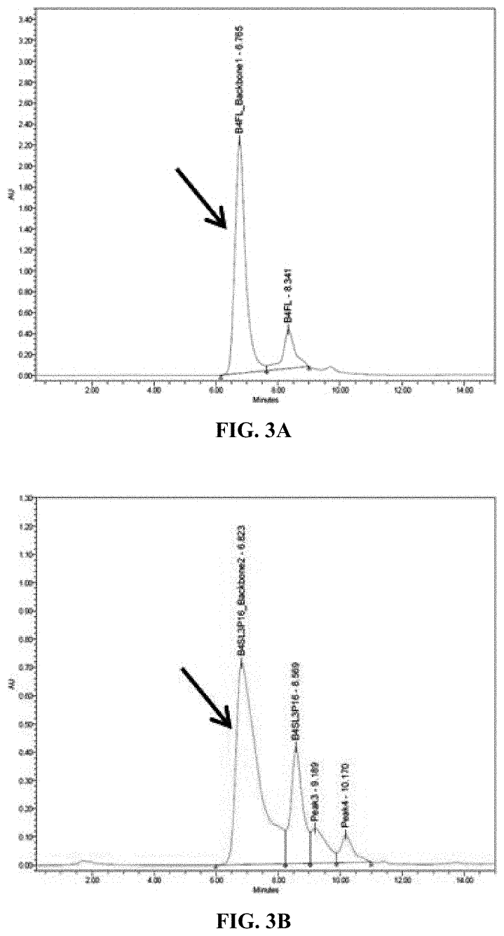

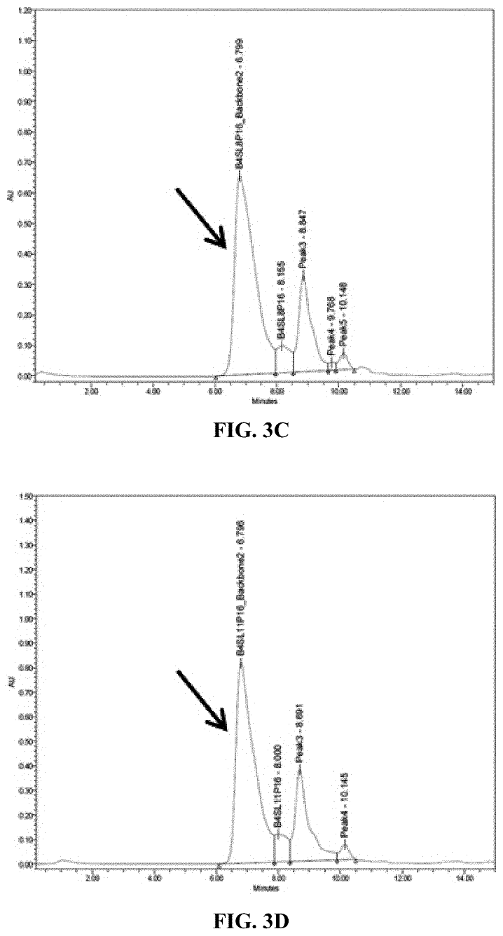

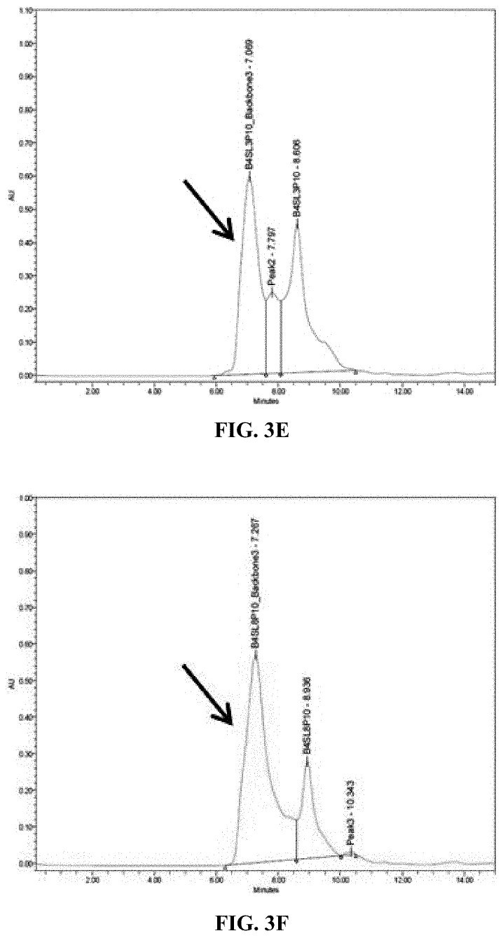

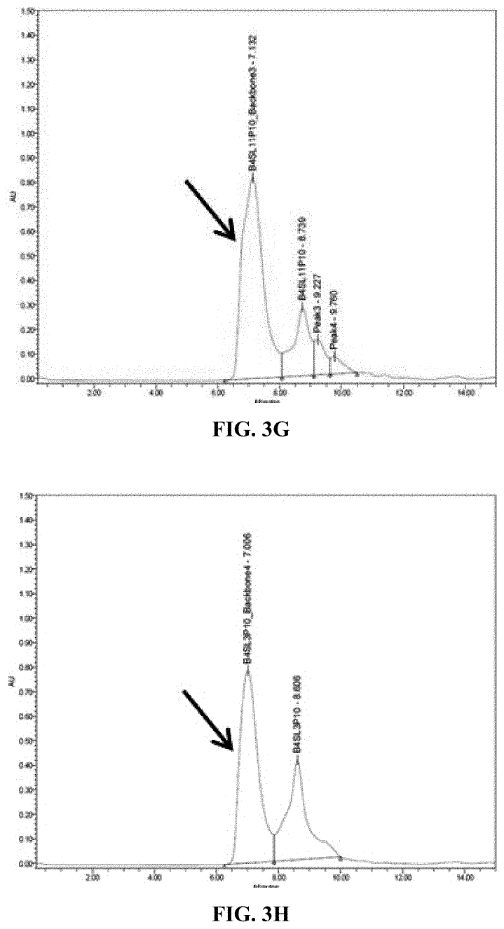

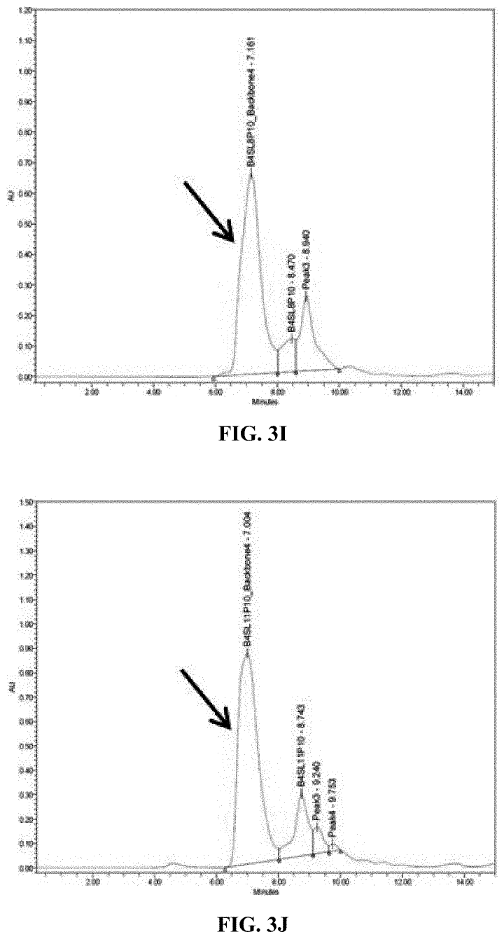

Tetramers of different backbone and aptamer sequences were formed by incubating 10 μM of each backbones with 60 μM of aptamers. The backbone and aptamer combinations and incubation conditions are shown in Table 4. Molar ratios of backbones and aptamers in tetramers formed from various backbone and aptamer sequences were examined using size exclusion chromatography and the results are shown in FIGS. 3A-3J. Multiple peaks were detected in chromatographs of several tetramer combinations suggesting that these combinations form partial structures (e.g., FIGS. 3B-3C). Partial structures include structures other than the desired tetramer such as trimer or dimer. A peak corresponding to a tetramer and a second peak corresponding to the free aptamers were detected, indicating formation of the desired aptamer tetramers. FIGS. 3A and 3H.

TABLE 4
Exemplary LAG-3 Aptamer Tetramer Synthesis Summary.

| | | | | | Theoretical | Final | FIG. |
|---|---|---|---|---|---|---|---|
| Backbone | Aptamer | Condition | | MERs | MW | Conc. | No. |
| Backbone R1 | B4_FL | 56° | C. | 368 | 121440 | 2.91 μM | 3A |
| Backbone R2 | | 16 | hr | | | | |
| R1_B10P16 | B4_SL3_P16 | 37° | C. | 308 | 101640 | 1.41 μM | 3B |
| R2_B10P16 | B4_SL8_P16 | 16 | hr | 268 | 88440 | 1.39 μM | 3C |
| | B4_SL11_P16 | | | 288 | 95040 | 1.5 μM | 3D |
| R1_B10P10 | B4_SL3_P10 | 37° | C. | 260 | 85800 | 1.68 μM | 3E |
| R2_B10P10 | B4_SL8_P10 | 16 | hr | 220 | 72600 | 1.92 μM | 3F |
| | B4_SL11_P10 | | | 240 | 79200 | 2.87 μM | 3G |
| R1_B18P10 | B4_SL3_P10 | 37° | C. | 276 | 91080 | 1.59 μM | 3H |
| R2_B18P10 | B4_SL8_P10 | 16 | hr | 236 | 77880 | 1.78 μM | 3I |
| | B4_SL11_P10 | | | 256 | 84480 | 1.89 μM | 3J |

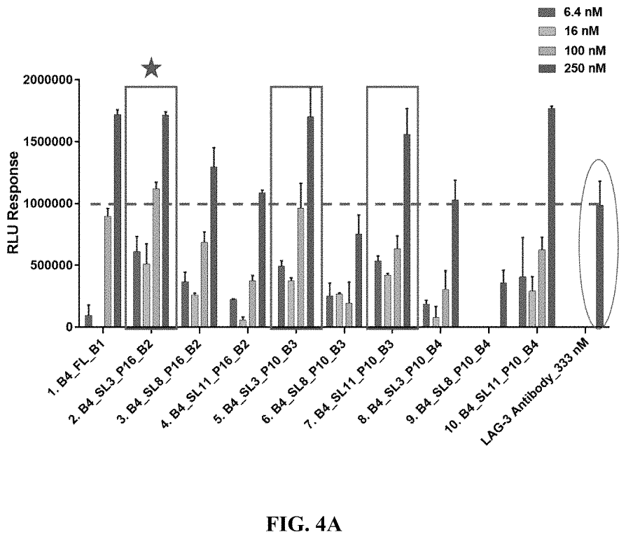

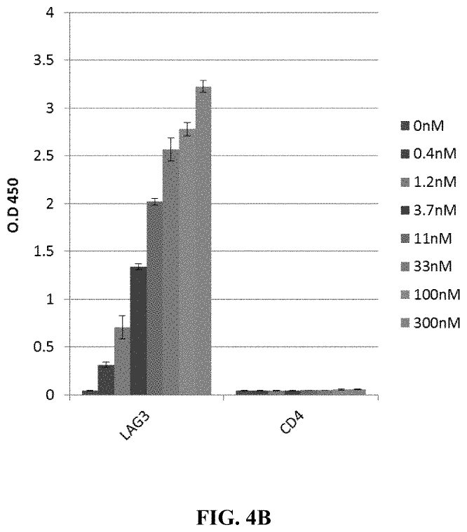

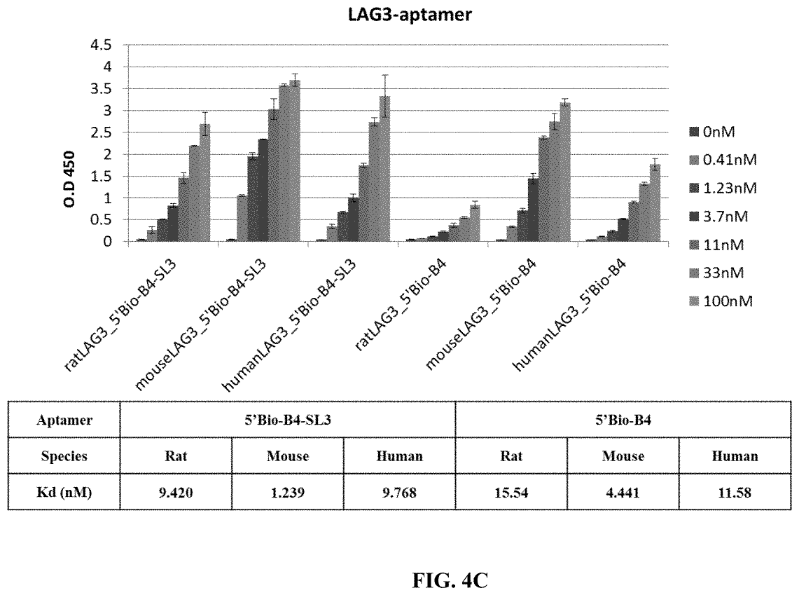

Next, the LAG-3 tetramers thus formed were tested for their activity in disrupting LAG-3 interaction with MHC-II using a LAG-3/MHC-II bioassay, in which the disruption of LAG-3 interaction with MHC-II is indicated by expression of a luciferase reporter. As shown in FIG. 4A, expression of the luciferase reporter was generally increased along with the increased concentrations of the tetramers. Several LAG-3 aptamer tetramers induced more luciferase expression than an anti-LAG-3 antibody even under the conditions that less LAG-3 tetramers (250 nM) were used in the assay relative to the anti-LAG-3 antibody (333 nM). FIG. 4A. These results suggest that the LAG-3 tetramers showed higher blocking activity toward LAG-3 binding to MHC-II than the anti-LAG-3 antibody.

To verify that LAG-3 tetramers bind to LAG-3, in vitro binding assays were performed using recombinant LAG-3 and LAG-3 tetramers. Binding of the LAG-3 tetramers to LAG-3 in a dose-dependent manner was observed. FIG. 4B. CD4 is structurally similar to LAG-3 and also binds to MHC-II. However, no binding was detected between LAG-3 tetramers and CD4, indicating that the binding between the LAG-3 aptamer tetramers and LAG-3 is specific. FIG. 4B.

Binding of LAG-3 aptamers (free or in tetramer form) to LAG-3 of different species (e.g., rat, mouse, and human) was also tested using the in vitro binding assays described herein. The results indicate that the LAG-3 aptamers can cross-react with LAG-3 from rat, mouse and human. FIG. 4C.

Taken together, these results demonstrate that LAG-3 aptamers, in free or tetramer form, bind to LAG-3 from various species, and that binding of the LAG-3 aptamers to LAG-3 inhibited LAG-3's interaction with MHC-II.

### Example 3: LAG-3 Tetramers Protected Mice Against Tumor Formation

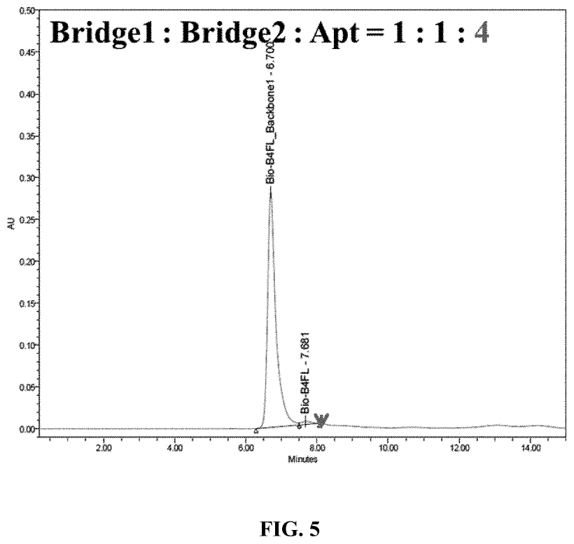

The anti-tumor activity of LAG-3 aptamers in tetrameric form was investigated as follows. Mice were subcutaneously inoculated with CT26 colon carcinoma cells to allow formation of tumor xenografts. The mice were then administered anti-PD-L1 antibody alone or in combination with LAG-3 aptamers in tetrameric form, or a vehicle control. LAG-3 tetramers were formed using backbones and the B4-SL3 aptamer sequence. Molar ratios of backbones to aptamers determined using size exclusion chromatography confirmed that LAG-3 tetramers with B4-SL3 aptamers (FIG. 5) were formed.

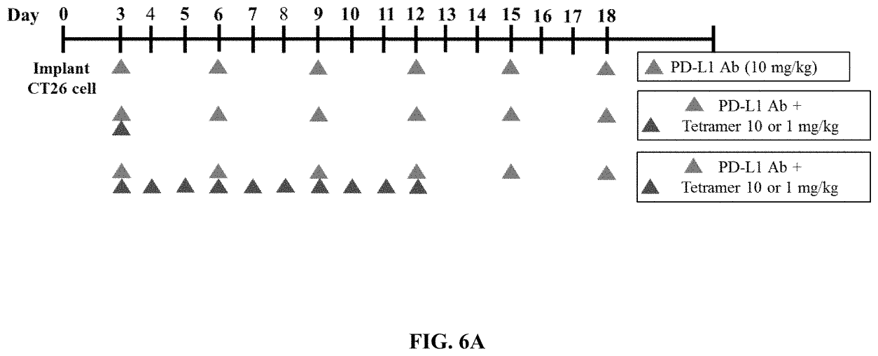

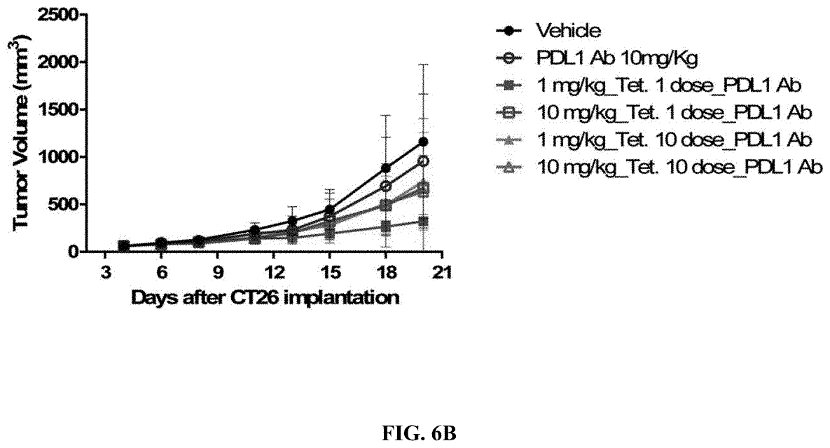

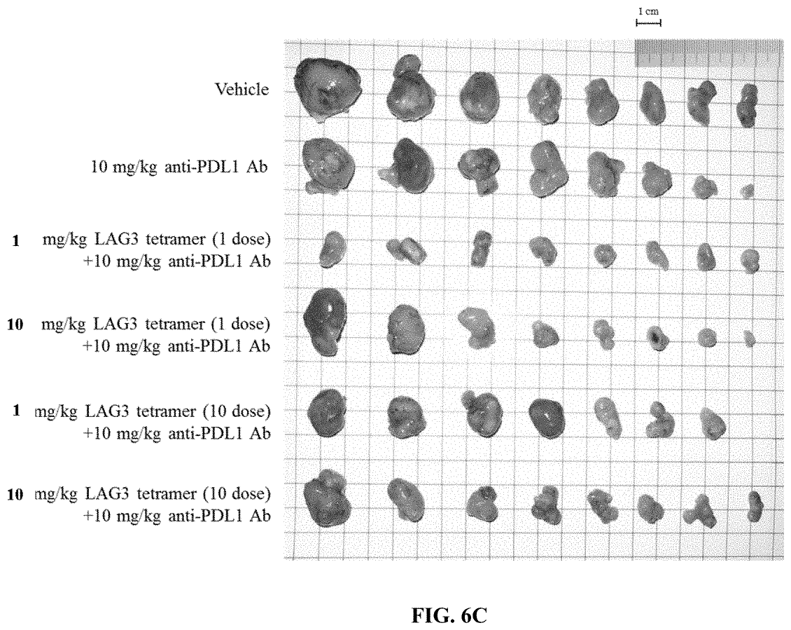

Mice were administered either 1 dose of an LAG-3 tetramers at day 3 or 10 consecutive doses of the LAG-3 tetramer on days 3-12, one dose per day. An illustration of the treatment regimen is provided in FIG. 6A. Mice treated with LAG-3 tetramer and PD-L1 antibody showed significantly reduced tumor growth compared to mice treated with PD-L1 antibody alone or vehicle control. FIG. 6B. Reduced tumor growth was observed in mice treated with 1 dose of LAG-3 aptamer compared to mice treated with 10 doses of LAG-3 aptamer, and greater reductions were observed using 1 mg/kg LAG-3 tetramer compared to dosing with 10 mg/kg LAG-3 tetramer. Surgically removed tumors from mice administered different treatments are shown in FIG. 6C.

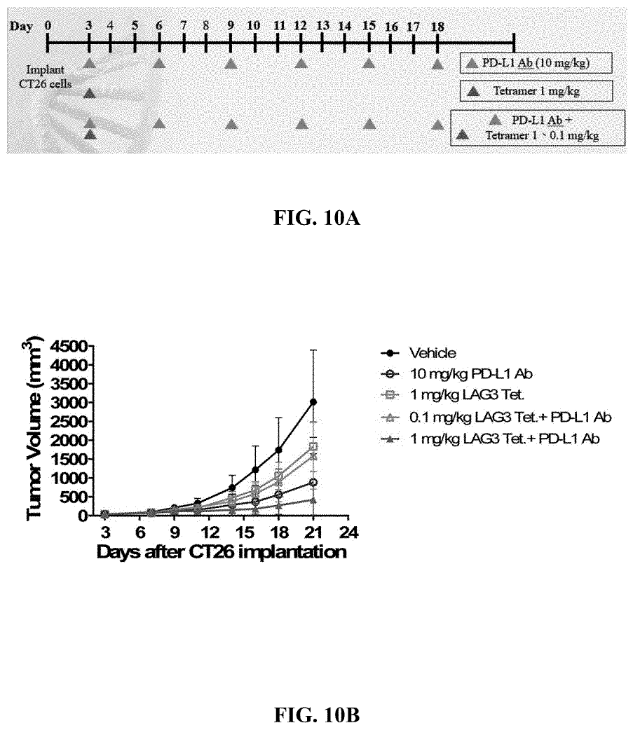

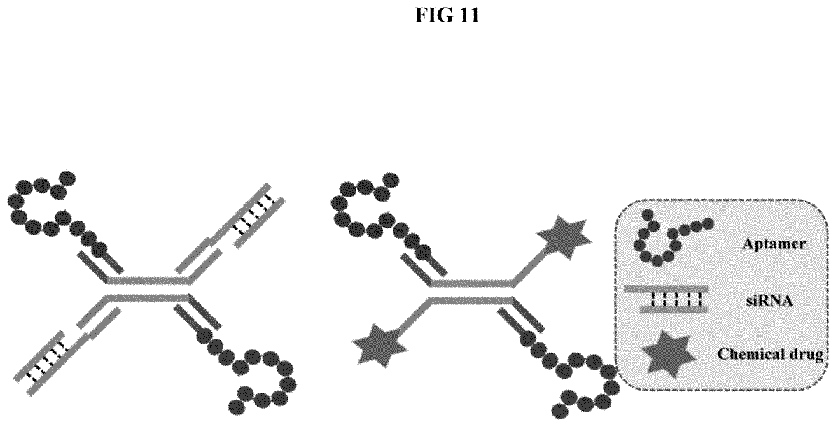

In a related study, mice were subcutaneously inoculated with CT26 colon carcinoma cells to allow formation of tumor xenografts. The mice were randomly assigned to 5 groups (each including 6 mice), each of which was treated with: (A) a vehicle control (6 injections with a 3-day interval followed by one final injection), (B) the anti-PD-L1 antibody (10 mg/kg; 6 injections with a 3-day interval), (C) the LAG-3 aptamers (1 mg/kg, a single dose), (D) the anti-PD-L1 antibody (10 mg/kg; 6 injections with a 3-day interval)+the LAG3 tetramer (1 mg/kg; a single dose), and (E) the anti-PD-L1 antibody (10 mg/kg; 6 injections with a 3-day interval) and the LAG3 tetramer (1 mg/kg; a single dose); following the administration regimen illustrated in FIG. 10A. As shown in FIG. 10B, co-administration of the anti-PD-L1 antibody and LAG3 tetramer significantly reduced tumor volumes in the xenograft mice as relative to the vehicle control. Surprisingly, a single dose of the LAG3 tetramer was sufficient to achieve the anti-tumor effects.

Taken together, these results indicate that LAG-3 tetramer protects against tumor formation of CT26 colon carcinoma cells.

### Example 4: Construction of LAG-3 Tetramers Using Identical Backbone Sequences Annealed Via Palindromic Sequences

LAG-3 tetramers were constructed using a backbone sequence having palindromic residues which allowed two identical backbone sequences to anneal via base pairing of the palindromic residues. Backbone sequences having 8 residues, 10 residues 12 residues and 16 palindromic residues were tested for tetramer formation by agarose gel analysis and size exclusion chromatography. Aptamer sequence and backbone sequences with palindromic residues in bold and underlined are shown in Table 5.

TABLE 5
Exemplary Aptamer and Bridge Sequences.

| | bp | Sequence |
|---|---|---|
| Aptamer | | |
| B4_SL3_ | 51 | GCTAACTGGGGGGGGTTAGTTCAATACATGC |
| P16 | | GGGCGCCACCGTGCTACAAC |
| | | (SEQ ID NO: 41) |
| Backbone | | |
| B8P16 | 50 | GTTGTAGCACGGTGGCTTTTTTCAGCTGATT |
| | | TTTGTTGTAGCACGGTGGC |
| | | (SEQ ID NO: 51) |
| B10P16 | 52 | GTTGTAGCACGGTGGCTTTTTATCAGCTGAT |
| | | TTTTTGTTGTAGCACGGTGGC |
| | | (SEQ ID NO: 52) |
| B12P16 | 54 | GTTGTAGCACGGTGGCTTTTTATATCGCGAT |
| | | ATTTTTTGTTGTAGCACGGTGGC |
| | | (SEQ ID NO: 53) |
| B8P16 | 58 | GTTGTAGCACGGTGGCTTTTTATATGACGCG |
| | | TCATATTTTTTGTTGTAGCACGGTGGC |
| | | (SEQ ID NO: 54) |

* Palindromic residues are shown in bold and underlined.

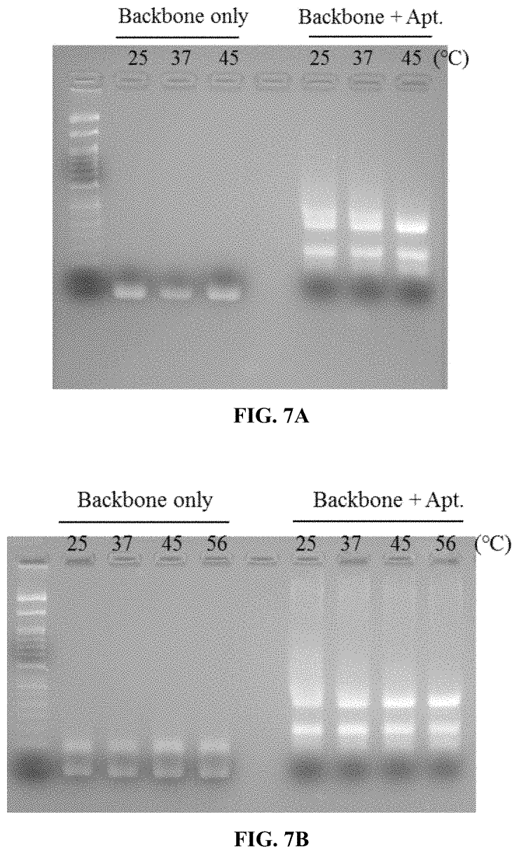

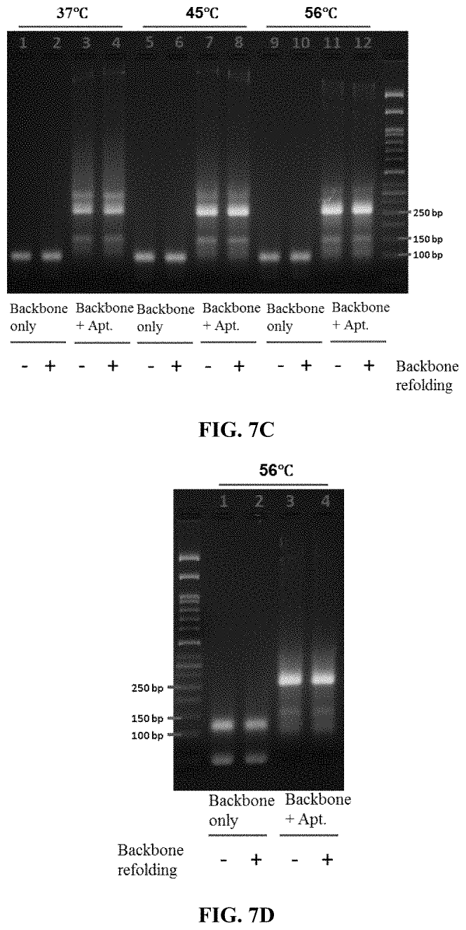

Backbone sequences having 8 palindromic residues (FIG. 7A) and 12 palindromic residues (FIG. 7C) showed a single band in the backbone only lane indicative of a duplex formed from annealing of two identical backbone sequences. By contrast, backbone sequences having 10 palindromic residues (FIG. 7B) and 16 palindromic residues (FIG. 7D) showed two bands in the backbone only lanes indicating that a duplex of identical backbone sequences was not formed for these sequences.

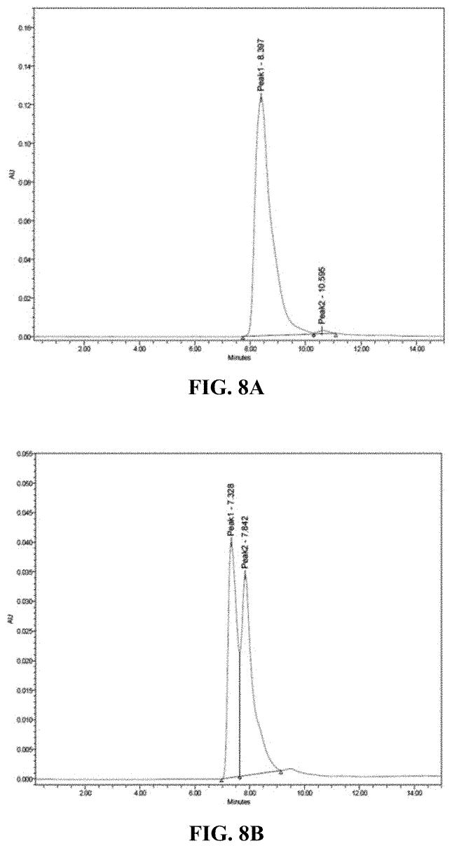

The above results were confirmed using size exclusion chromatograph. A predominate single peak in the chromatograph of LAG-3 tetramer formed using a backbone sequence having 12 palindromic residues (FIG. 8A) was observed, suggesting that a single tetrameric structure was formed. Two similarly sized peaks were observed in the chromatograph of LAG-3 tetramer formed using a backbone sequence having 8 palindromic residues (FIG. 8B) suggesting that a duplex of identical backbone sequences was not formed. These results demonstrated that LAG-3 tetramers were formed using identical backbone sequences annealed via 12 palindromic residues.

Next, annealing of backbone sequences with different 12 residue palindromic sequences were analyzed by agarose gel and free energy analysis. Various combinations of a palindromic sequence having a general formula of (A/T)₄(C/G)₄(A/T)₄ were examined.

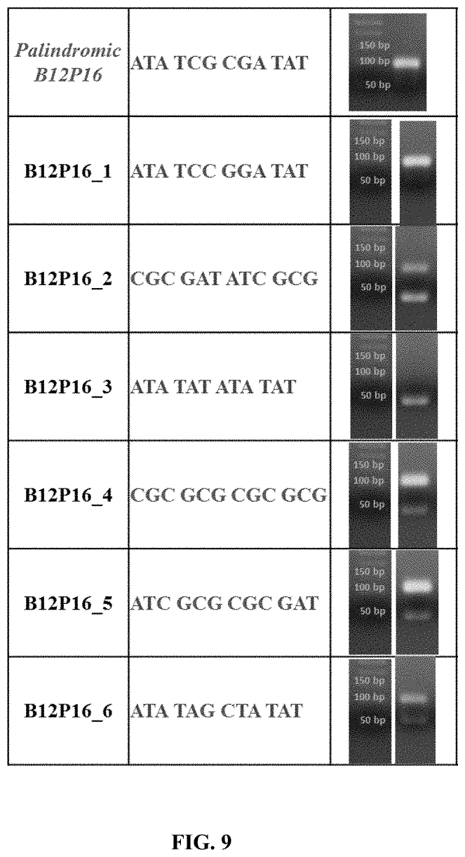

As shown in FIG. 9 and Table 6, palindromic sequences ATATCGCGATAT (SEQ ID NO:17) and ATATCCGGATAT (SEQ ID NO:18) showed over 90% dimer formation by agarose gel analysis, which was highest among the tested sequences. Palindromic sequences and percent dimer and/or percent monomer formation as detected by agarose gel analysis are shown in Table 6 below.

TABLE 6
Exemplary Palindromic Sequences and Agarose
Gel Analysis Results.

| | | Agarose Gel |
|---|---|---|
| Backbone | Sequence | Analysis Results |
| B12P16 | ATA TCG CGA TAT | Over 90% dimer |
| | (SEQ ID NO: 17) | |
| B12P16_1 | ATA TCC GGA TAT | Over 90% dimer |
| | (SEQ ID NO: 18) | |
| B12P16_2 | CGC GAT ATC GCG | About 80% monomer |
| | (SEQ ID NO: 19) | (stem-loop) |
| B12P16_3 | ATA TAT ATA TAT | About 90% monomer |
| | (SEQ ID NO: 20) | (stem-loop) |
| B12P16_4 | CGC GCG CGC GCG | About 60% dimer, |
| | (SEQ ID NO: 21) | 40% monomer |
| B12P16_5 | ATC GCG CGC GAT | About 60% dimer, |
| | (SEQ ID NO: 22) | 40% monomer |
| B12P16_6 | ATA TAG CTA TAT | About 60% dimer, |
| | (SEQ ID NO: 23) | 40% monomer |

Similar results were obtained by free energy analysis of palindromic sequences. As shown in Table 7 below, palindromic sequences ATATCGCGATAT (SEQ ID NO:17) and ATATCCGGATAT (SEQ ID NO:18) were favored for formation of a dimer among the analyzed sequences based on free energy calculations.

TABLE 7
Free Energy Analysis of Exemplary Palindromic
Sequences.

| | | Monomer | Dimer | Difference |
|---|---|---|---|---|
| Backbone | Sequence | (ΔG) | (ΔG) | (ΔG) |
| B12P16 | ATA TCG CGA TAT | −0.61 | −7.40 | −6.79 |
| | (SEQ ID NO: 17) | | | |
| B12P16_1 | ATA TCC GGA TAT | −0.95 | −6.67 | −5.72 |
| | (SEQ ID NO: 18) | | | |
| B12P16_2 | CGC GAT ATC GCG | −4.61 | −12.22 | −7.61 |
| | (SEQ ID NO: 19) | | | |
| B12P16_3 | ATA TAT ATA TAT | −0.61 | −2.71 | −2.10 |
| | (SEQ ID NO: 20) | | | |
| B12P16_4 | CGC GCG CGC GCG | −4.61 | −18.34 | −13.73 |
| | (SEQ ID NO: 21) | | | |
| B12P16_5 | ATC GCG CGC GAT | −2.40 | −13.30 | −10.90 |
| | (SEQ ID NO: 22) | | | |
| B12P16_6 | ATA TAG CTA TAT | −1.10 | −4.18 | −3.08 |
| | (SEQ ID NO: 23) | | | |
| B12P16_7 | TTA TGC GCA TAA | −0.61 | −7.37 | −6.76 |
| | (SEQ ID NO: 55) | | | |
| B12P16_8 | ATA TGG CCA TAT | −1.35 | −7.04 | −5.69 |
| | (SEQ ID NO: 56) | | | |
| B12P16_9 | TTA AGG CCT TAA | −1.61 | −6.54 | −4.93 |
| | (SEQ ID NO: 57) | | | |
| B22P16_10 | AAA AGC GCT TTT | −1.70 | −8.75 | −7.05 |
| | (SEQ ID NO: 58) | | | |
| B22P16_11 | TAA AGC GCT TTA | −1.70 | −7.87 | −6.17 |
| | (SEQ ID NO: 59) | | | |
| B12P16_12 | TAT ACC GGT ATA | −2.21 | −6.31 | −4.1 |
| | (SEQ ID NO: 60) | | | |

Taken together, these results demonstrate formation of a LAG-3 tetramer using identical backbone sequences annealed via 12 palindromic residues having a general formula of (A/T)₄(C/G)₄(A/T)₄.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A nucleic acid aptamer, comprising a nucleotide motif of:
(a) GX₁GGGX₂GGTX₃A (SEQ ID NO:1), in which each of X₁ and X₂ are independently G, C, or absent, and X₃ is T or C, or
(b) L-(G)ₙ-L′, in which n is an integer of 5-9 inclusive, and L and L′ are nucleotide segments having complementary sequences;
wherein the nucleic acid aptamer binds human lymphocyte activation gene 3 (LAG-3).

2. The nucleic acid aptamer of claim 1, wherein the nucleotide motif (a) is GGGGGGGGTTA (SEQ ID NO:2).

3. The nucleic acid aptamer of claim 1 or claim 2, wherein the nucleic acid aptamer comprises a nucleotide sequence at least 85% identical to one of the following nucleotide sequences:

| (SEQ ID NO: 8) | |
|---|---|
| (i) | TGGGGGGGGTTAGTTCAATACATGCGGGCG; |
| (SEQ ID NO: 9) | |
| (ii) | TGGGGGGGGGTTAGACTTACACTCTTATTCG; |
| (SEQ ID NO: 10) | |
| (iii) | AGAGGGGGGGGTTAGCTGCTTTAACTCATG; |
| and | |
| (SEQ ID NO: 11) | |
| (iv) | AGGGGGGGGGTTACTGCGCATGTATCTCAG. |

4. The nucleic acid aptamer of claim 3, wherein the nucleic acid aptamer comprises a nucleotide sequence at least 90% identical to one of the following nucleotide sequences:

| (SEQ ID NO: 8) | |
|---|---|
| (i) | TGGGGGGGGTTAGTTCAATACATGCGGGCG; |
| (SEQ ID NO: 9) | |
| (ii) | TGGGGGGGGGTTAGACTTACACTCTTATTCG; |
| (SEQ ID NO: 10) | |
| (iii) | AGAGGGGGGGGTTAGCTGCTTTAACTCATG; |
| and | |
| (SEQ ID NO: 11) | |
| (iv) | AGGGGGGGGGTTACTGCGCATGTATCTCAG. |

5. The nucleic acid aptamer of claim 4, wherein the nucleic acid aptamer comprises a nucleotide sequence at least 95% identical to one of the following nucleotide sequences:

| (SEQ ID NO: 8) | |
|---|---|
| (i) | TGGGGGGGGTTAGTTCAATACATGCGGGCG; |
| (SEQ ID NO: 9) | |
| (ii) | TGGGGGGGGGTTAGACTTACACTCTTATTCG; |
| (SEQ ID NO: 10) | |
| (iii) | AGAGGGGGGGGTTAGCTGCTTTAACTCATG; |
| and | |
| (SEQ ID NO: 11) | |
| (iv) | AGGGGGGGGGTTACTGCGCATGTATCTCAG. |

6. The nucleic acid aptamer of claim 5, wherein the nucleic acid aptamer comprises one of the following nucleotide sequences:

| (SEQ ID NO: 8) | |
|---|---|
| (i) | TGGGGGGGGTTAGTTCAATACATGCGGGCG; |
| (SEQ ID NO: 9) | |
| (ii) | TGGGGGGGGGTTAGACTTACACTCTTATTCG; |
| (SEQ ID NO: 10) | |
| (iii) | AGAGGGGGGGGTTAGCTGCTTTAACTCATG; |
| and | |
| (SEQ ID NO: 11) | |
| (iv) | AGGGGGGGGGTTACTGCGCATGTATCTCAG. |

7. The nucleic acid aptamer of claim 2, wherein the nucleic acid aptamer comprises the nucleotide sequence of (i) TGGGGGGGGTTAGTTCAATACATG (SEQ ID:12).

8. The nucleic acid aptamer of claim 7, wherein the nucleic acid aptamer is selected from the group consisting of:
(SEQ ID NO: 13)

| | |
|---|---|
| (a) | TCCCTACGGCGCTAACTGGGGGGGGTTAGTTCAATACATGCGGG |
| | CGGCCACCGTGCTACAAC; |

(SEQ ID NO: 14)

| | |
|---|---|
| (b) | ACGGCGCTAACTGGGGGGGGTTAGTTCAATACATG; |

(SEQ ID NO: 15)

| | |
|---|---|
| (c) | GCTAACTGGGGGGGGTTAGTTCAATACATGCGGGC; |
| and | |

(SEQ ID NO: 16)

| | |
|---|---|
| (d) | CTGGGGGGGGTTAGTTCAATACATGCGGGCGGCCA. |

9. The nucleic acid aptamer of any one of claims 1-8, wherein the nucleic acid aptamer consists of about 35-100 nucleotides.

10. The nucleic acid aptamer of any one of claims 1-9, wherein the nucleic acid aptamer consists of about 35-70 nucleotides.

11. The nucleic acid aptamer of claim 1, wherein the nucleic acid aptamer comprises the motif (b) and wherein L and L′ in motif (b) each have 5-8 nucleotides.

12. A multimeric nucleic acid aptamer, comprising a first polynucleic acid, a first nucleic acid aptamer, and a second nucleic acid aptamer, wherein the first polynucleic acid comprises a nucleotide sequence formula of 5′-X-L₁-Y-L₂-Z-3′, in which each of X and Z is a nucleotide segment complementary to a portion of the first nucleic acid aptamer and/or the second nucleic acid aptamer, each of L₁ and L₂ independently is a linker or is absent, and Y is a nucleotide segment having a palindromic sequence; and wherein the first nucleic acid aptamer and the second nucleic acid aptamer form duplexes with the X and Z regions of the first polynucleic acid.

13. The multimeric nucleic acid aptamer of claim 12, further comprising a second polynucleic acid, a third nucleic acid aptamer, and a fourth nucleic acid aptamer, wherein the second polynucleic acid comprises a nucleotide sequence formula of 5′-X′-L₁′-Y-L₂′-Z′-3′, in which each of X′ and Z′ is a nucleotide segment complementary to a portion of the third nucleic acid aptamer and/or the fourth nucleic acid aptamer, each of L₁′ and L₂′ independently is a linker or is absent, and Y is the nucleotide segment having the palindromic sequence; wherein the third nucleic acid aptamer and the fourth nucleic acid aptamer form duplexes with the X′ and Z′ regions of the second polynucleic acid, and wherein the first polynucleic acid and the second polynucleic acid form a duplex at the palindromic sequence region.

14. The multimeric nucleic acid aptamer of claim 12 or claim 13, wherein the L₁, L₂, L₁′, and/or L₂′ is a linker, which optionally is a polyA or polyT segment.

15. The multimeric nucleic acid aptamer of claim 14, wherein the polyA or polyT segment consists of 4-10 nucleotides.

16. The multimeric nucleic acid aptamer of any one of claims 12-15, wherein the palindromic sequence consists of 8, 10, 12, 14, or 16 nucleotides.

17. The multimeric nucleic acid aptamer of claim 16, wherein the palindromic sequence is (A/T)₄(C/G)₄(A/T)₄.

18. The multimeric nucleic acid aptamer of any one of claims 12-17, wherein the X and X′, L₁ and L₁′, L₂ and L₂′, and/or Z and Z′ in the first polynucleic acid and the second polynucleic acid are identical.

19. The multimeric nucleic acid aptamer of any one of claims 12-18, wherein at least two of the first nucleic acid aptamer, the second nucleic acid aptamer, the third nucleic acid aptamer and the fourth nucleic acid aptamer are specific to a same target molecule of interest.

20. The multimeric nucleic acid aptamer of claim 19, wherein at least two of the first nucleic acid aptamer, the second nucleic acid aptamer, the third nucleic acid aptamer and the fourth nucleic acid aptamer are the same.

21. The multimeric nucleic acid aptamer of claim 20, wherein all of the first nucleic acid aptamer, the second nucleic acid aptamer, the third nucleic acid aptamer and the fourth nucleic acid aptamer are the same.

22. The multimeric nucleic acid aptamer of any one of claims 12-18, wherein at least two of the first nucleic acid aptamer, the second nucleic acid aptamer, the third nucleic acid aptamer and the fourth nucleic acid aptamer are specific to different target molecules of interest.

23. The multimeric nucleic acid aptamer of claim 22, wherein at least two of the first nucleic acid aptamer, the second nucleic acid aptamer, the third nucleic acid aptamer and the fourth nucleic acid aptamer are different aptamers.

24. The multimeric nucleic acid aptamer of claim 23, wherein all of the first nucleic acid aptamer, the second nucleic acid aptamer, the third nucleic acid aptamer and the fourth nucleic acid aptamer are different aptamers.

25. A multimeric nucleic acid complex, comprising a first polynucleic acid comprising a nucleotide sequence formula of 5′-X-L₁-Y-L₂-Z-3′, in which X represents a first nucleic acid, Z represents a second nucleic acid, each of L₁ and L₂ independently is a linker or is absent, and Y is a nucleotide segment having a palindromic sequence.

26. The multimeric nucleic acid complex of claim 25, further comprising a second polynucleic acid, which comprises a nucleotide sequence formula of 5′-X′-L₁′-Y′-L₂′-Z′-3′, in which X′ represents a third nucleic acid, Z′ represents a fourth nucleic acid, each of L₁′ and L₂′ independently is a linker or is absent, and Y is the nucleotide segment having the palindromic sequence; wherein the first polynucleic acid and the second polynucleic acid form a duplex at the palindromic sequence region.

27. The multimeric nucleic acid complex of claim 25 or claim 26, wherein the first nucleic acid, the second nucleic acid, the third nucleic acid, the fourth nucleic acid, or a combination thereof are nucleic acid aptamers.

28. The multimeric nucleic acid complex of claim 25 or claim 26, wherein the first nucleic acid, the second nucleic acid, the third nucleic acid, the fourth nucleic acid, or a combination thereof are antisense oligonucleotides.

29. The multimeric nucleic acid complex of claim 25 or claim 26, wherein the first nucleic acid, the second nucleic acid, the third nucleic acid, the fourth nucleic acid, or a combination thereof are siRNAs.

30. The multimeric nucleic acid complex of any one of claims 25-29, wherein at least one of the first nucleic acid, the second nucleic acid, the third nucleic acid, and the fourth nucleic acid is conjugated to a therapeutic agent.

31. The multimeric nucleic acid complex of claim 30, wherein the therapeutic agent is a small molecule drug, a peptide drug, or a protein drug.

32. The multimeric nucleic acid complex of any one of claims 25-31, wherein at least two of the first nucleic acid, the second nucleic acid, the third nucleic acid and the fourth nucleic acid are specific to a same target molecule of interest.

33. The multimeric nucleic acid complex of claim B32, wherein at least two of the first nucleic acid, the second nucleic acid, the third nucleic acid and the fourth nucleic acid are identical nucleic acid aptamers.

34. The multimeric nucleic acid complex of claim 32, wherein all of the first nucleic acid, the second nucleic acid, the third nucleic acid and the fourth nucleic acid are identical nucleic acid aptamers.

35. The multimeric nucleic acid complex of any one of claims 25-31, wherein at least two of the first nucleic acid, the second nucleic acid, the third nucleic acid and the fourth nucleic acid are specific to different target molecules of interest.

36. The multimeric nucleic acid complex of claim 35, wherein at least two of the first nucleic acid, the second nucleic acid, the third nucleic acid and the fourth nucleic acid are different nucleic acid aptamers.

37. The multimeric nucleic acid complex of claim 36, wherein all of the first nucleic acid, the second nucleic acid, the third nucleic acid and the fourth nucleic acid are different nucleic acid aptamers.

38. The multimeric nucleic acid complex of any one of claims 12-37, wherein at least one of the first nucleic acid, the second nucleic acid, the third nucleic acid, and the fourth nucleic acid is a nucleic acid aptamer set forth in any one of claims 1-10.

39. The multimeric nucleic acid complex of any one of claims 12-37, wherein at least one of the first nucleic acid, the second nucleic acid, the third nucleic acid, and the fourth nucleic acid is a nucleic acid aptamer, and at least one of the other nucleic acids is an antisense oligonucleotide, a siRNA or is conjugated to the therapeutic agent.

40. The multimeric nucleic acid complex of any one of claim 25 or claim 26, wherein the multimeric nucleic acid complex further comprises a nucleic acid set, which comprises a fifth nucleic acid, a six nucleic acid, a seventh nucleic acid, an eighth nucleic acid, or a combination thereof, wherein each nucleic acid of the nucleic acid set comprises a portion that is complementary to the first nucleic acid, the second nucleic acid, the third nucleic acid, or the fourth nucleic acid and forms duplex with the first nucleic acid, the second nucleic acid, the third nucleic acid, or the fourth nucleic acid.

41. The multimeric nucleic acid complex of claim 40, wherein the fifth nucleic acid, the sixth nucleic acid, the seventh nucleic acid, the eighth nucleic acid, or a combination thereof comprises a nucleic acid aptamer.

42. The multimeric nucleic acid complex of claim 40, wherein the fifth nucleic acid, the sixth nucleic acid, the seventh nucleic acid, the eighth nucleic acid, or a combination thereof comprises an antisense oligonucleotide.

43. The multimeric nucleic acid complex of claim 40, wherein the fifth nucleic acid, the sixth nucleic acid, the seventh nucleic acid, the eighth nucleic acid, or a combination thereof comprises an siRNA.

44. The multimeric nucleic acid complex of any one of claims B23-B26, wherein at least one of the fifth nucleic acid, the sixth nucleic acid, the seventh nucleic acid, and the eighth nucleic acid is conjugated to a therapeutic agent.

45. The multimeric nucleic acid complex of claim 44, wherein the therapeutic agent is a small molecule drug, a peptide drug, or a protein drug.

46. The multimeric nucleic acid complex of any one of claims 40-45, wherein at least two of the fifth nucleic acid, the sixth nucleic acid, the seventh nucleic acid and the eighth nucleic acid are specific to a same target molecule of interest.

47. The multimeric nucleic acid complex of claim 46, wherein at least two of the fifth nucleic acid, the sixth nucleic acid, the seventh nucleic acid and the eighth nucleic acid comprise an identical nucleic acid aptamer.

48. The multimeric nucleic acid complex of claim 46, wherein all of the fifth nucleic acid, the sixth nucleic acid, the seventh nucleic acid aptamer and the eighth nucleic acid comprise an identical nucleic acid aptamer.

49. The multimeric nucleic acid complex of any one of claims 40-45, wherein at least two of the fifth nucleic acid, the sixth nucleic acid, the seventh nucleic acid and the eighth nucleic acid are specific to different target molecules of interest.

50. The multimeric nucleic acid complex of claim 49, wherein at least two of the fifth nucleic acid, the sixth nucleic acid, the seventh nucleic acid and the eighth nucleic acid comprise different nucleic acid aptamers.

51. The multimeric nucleic acid complex of claim 50, wherein all of the fifth nucleic acid, the sixth nucleic acid, the seventh nucleic acid and the eighth nucleic acid comprise different nucleic acid aptamers.

52. The multimeric nucleic acid complex of any one of claims 40-51, wherein at least one of the fifth nucleic acid, the sixth nucleic acid, the seventh nucleic acid, and the eighth nucleic acid is a nucleic acid aptamer set forth in any one of claims 1-10.

53. The multimeric nucleic acid complex of any one of claims 40-51, wherein at least one of the fifth nucleic acid, the sixth nucleic acid, the seventh nucleic acid, and the eighth nucleic acid comprises a nucleic acid aptamer, and at least one of the other nucleic acids comprises an antisense oligonucleotide, an siRNA or is conjugated to the therapeutic agent.

54. The multimeric nucleic acid of any one of claims 25-53, wherein the L₁, L₂, L₁′, and/or L₂′ is a linker, which optionally is a polyA or polyT segment.

55. The multimeric nucleic acid of claim 54, wherein the polyA or polyT segment consists of 4-10 nucleotides.

56. The multimeric nucleic acid of any one of claims 25-55, wherein the palindromic sequence consists of 8, 10, 12, 14, or 16 nucleotides.

57. The multimeric nucleotide of claim 56, wherein the palindromic sequence is (A/T)₄(C/G)₄(A/T)₄.

58. The multimeric nucleic acid of any one of claims 25-57, wherein the L₁, and L₁′, and/or L₂ and L₂′ in the first polynucleic acid and the second polynucleic acid are identical.

59. A pharmaceutical composition, comprising a nucleic acid aptamer of any one of claims 1-11, and/or a multimeric nucleic acid aptamer of any one of claims 12-58, and a pharmaceutically acceptable carrier.

60. A method for modulating immune responses, the method comprising administering to a subject in need thereof a pharmaceutical composition of claim 59.

61. The method of claim 60, wherein the subject is a human patient having, suspected of having, or at risk for a cancer.

62. The method of claim 61, wherein the cancer is selected from the group consisting of lung cancer, melanoma, colorectal cancer, renal-cell cancer, urothelial carcinoma, and Hodgkin's lymphoma.

63. The method of any one of claims 60-62, wherein the pharmaceutical composition is administered to the subject intravenously.

64. The method of any one of claims 61-63, wherein the pharmaceutical composition is in an amount sufficient to enhance T cell activity and/or inhibit cancer growth in the subject.

65. The method of any one of claims 61-64, wherein the pharmaceutical composition is administered to the subject in only one dose.

66. A method for detecting presence of LAG-3-positive cells, comprising:
(i) contacting cells suspected of expressing LAG-3 with a nucleic acid aptamer of any one of claims 1-11 or a multimeric nucleic acid aptamer of any one of claims 12-58, wherein the nucleic acid aptamer or multimeric nucleic acid aptamer is conjugated to a detection agent; and
(ii) measuring a signal released from the detection agent conjugated to the nucleic acid aptamer or multimeric nucleic acid aptamer that is bound to a cell; wherein intensity of the signal is indicative of presence or level of LAG-3-positive cells.

67. The method of claim 66, wherein the contacting step (i) is performed by administering the nucleic acid aptamer or multimeric nucleic acid aptamer to a subject in need thereof.


SEQUENCE LISTING (60 sequences)

SEQ ID NO: 1 - DNA, 11 bp, Artificial Sequence
  synthetic
  misc_feature (2)..(2): n is g, c, or absent
  misc_feature (6)..(6): n is g, c, or absent
  misc_feature (10)..(10): n is t or c
gngggnggtna

SEQ ID NO: 2 - DNA, 11 bp, Artificial Sequence
  synthetic
ggggggggtta

SEQ ID NO: 3 - DNA, 5 bp, Artificial Sequence
  synthetic
ggggg

SEQ ID NO: 4 - DNA, 6 bp, Artificial Sequence
  synthetic
gggggg

SEQ ID NO: 5 - DNA, 7 bp, Artificial Sequence
  synthetic
ggggggg

SEQ ID NO: 6 - DNA, 8 bp, Artificial Sequence
  synthetic
gggggggg

SEQ ID NO: 7 - DNA, 9 bp, Artificial Sequence
  synthetic
ggggggggg

SEQ ID NO: 8 - DNA, 30 bp, Artificial Sequence
  synthetic
tggggggggttagttcaatacatgcgggcg

SEQ ID NO: 9 - DNA, 31 bp, Artificial Sequence
  synthetic
tgggggggggttagacttacactcttattcg

SEQ ID NO: 10 - DNA, 30 bp, Artificial Sequence
  synthetic
agaggggggggttagctgctttaactcatg

SEQ ID NO: 11 - DNA, 30 bp, Artificial Sequence
  synthetic
agggggggggttactgcgcatgtatctcag

SEQ ID NO: 12 - DNA, 24 bp, Artificial Sequence
  synthetic
tggggggggttagttcaatacatg

SEQ ID NO: 13 - DNA, 62 bp, Artificial Sequence
  synthetic
tccctacggcgctaactggggggggttagttcaatacatgcgggcggccaccgtgctacaac

SEQ ID NO: 14 - DNA, 35 bp, Artificial Sequence
  synthetic
acggcgctaactggggggggttagttcaatacatg

SEQ ID NO: 15 - DNA, 35 bp, Artificial Sequence
  synthetic
gctaactggggggggttagttcaatacatgcgggc

SEQ ID NO: 16 - DNA, 35 bp, Artificial Sequence
  synthetic
ctggggggggttagttcaatacatgcgggcggcca

SEQ ID NO: 17 - DNA, 12 bp, Artificial Sequence
  synthetic
atatcgcgatat

SEQ ID NO: 18 - DNA, 12 bp, Artificial Sequence
  synthetic
atatccggatat

SEQ ID NO: 19 - DNA, 12 bp, Artificial Sequence
  synthetic
cgcgatatcgcg

SEQ ID NO: 20 - DNA, 12 bp, Artificial Sequence
  synthetic
atatatatatat

SEQ ID NO: 21 - DNA, 12 bp, Artificial Sequence
  synthetic
cgcgcgcgcgcg

SEQ ID NO: 22 - DNA, 12 bp, Artificial Sequence
  synthetic
atcgcgcgcgat

SEQ ID NO: 23 - DNA, 12 bp, Artificial Sequence
  synthetic
atatagctatat

SEQ ID NO: 24 - DNA, 9 bp, Artificial Sequence
  synthetic
ggggggtta

SEQ ID NO: 25 - DNA, 10 bp, Artificial Sequence
  synthetic
gggggggtta

SEQ ID NO: 26 - DNA, 12 bp, Artificial Sequence
  synthetic
gggggggggtta

SEQ ID NO: 27 - DNA, 10 bp, Artificial Sequence
  synthetic
ggggggttaa

SEQ ID NO: 28 - DNA, 10 bp, Artificial Sequence
  synthetic
gggggggtta

SEQ ID NO: 29 - DNA, 11 bp, Artificial Sequence
  synthetic
ggggggggtta

SEQ ID NO: 30 - DNA, 32 bp, Artificial Sequence
  synthetic
tccctacggcgctaactccctacggcgctaac

SEQ ID NO: 31 - DNA, 16 bp, Artificial Sequence
  synthetic
gccaccgtgctacaac

SEQ ID NO: 32 - DNA, 63 bp, Artificial Sequence
  synthetic
tccctacggcgctaactgggggggggttagacttacactcttattcggccaccgtgctacaac

SEQ ID NO: 33 - DNA, 62 bp, Artificial Sequence
  synthetic
tccctacggcgctaacagaggggggggttagctgctttaactcatggccaccgtgctacaac

SEQ ID NO: 34 - DNA, 62 bp, Artificial Sequence
  synthetic
tccctacggcgctaacagggggggggttactgcgcatgtatctcaggccaccgtgctacaac

SEQ ID NO: 35 - DNA, 8 bp, Artificial Sequence
  synthetic
tcagctga

SEQ ID NO: 36 - DNA, 10 bp, Artificial Sequence
  synthetic
atcagctgat

SEQ ID NO: 37 - DNA, 12 bp, Artificial Sequence
  synthetic
atatcgcgatat

SEQ ID NO: 38 - DNA, 16 bp, Artificial Sequence
  synthetic
atatgacgcgtcatat

SEQ ID NO: 39 - DNA, 35 bp, Artificial Sequence
  synthetic
gggggttagttcaatacatgcgggcggccaccgtg

SEQ ID NO: 40 - DNA, 35 bp, Artificial Sequence
  synthetic
ttagttcaatacatgcgggcggccaccgtgctaca

SEQ ID NO: 41 - DNA, 51 bp, Artificial Sequence
  synthetic
gctaactggggggggttagttcaatacatgcgggcgccaccgtgctacaac

SEQ ID NO: 42 - DNA, 46 bp, Artificial Sequence
  synthetic
gctaactggggggggttagttcaatacatggccaccgtgctacaac

SEQ ID NO: 43 - DNA, 41 bp, Artificial Sequence
  synthetic
gctaactggggggggttagttcaatgccaccgtgctacaac

SEQ ID NO: 44 - DNA, 45 bp, Artificial Sequence
  synthetic
gctaactggggggggttagttcaatacatgcgggcgtgctacaac

SEQ ID NO: 45 - DNA, 40 bp, Artificial Sequence
  synthetic
gctaactggggggggttagttcaatacatggtgctacaac

SEQ ID NO: 46 - DNA, 35 bp, Artificial Sequence
  synthetic
gctaactggggggggttagttcaatgtgctacaac

SEQ ID NO: 47 - DNA, 60 bp, Artificial Sequence
  synthetic
gttgtagcacggtggctttttatttaggtgacactatagtttttgttgtagcacggtggc

SEQ ID NO: 48 - DNA, 52 bp, Artificial Sequence
  synthetic
gttgtagcacggtggctttttaggtgacacttttttgttgtagcacggtggc

SEQ ID NO: 49 - DNA, 80 bp, Artificial Sequence
  synthetic
gttgtagcactttttaggtgacacttttttgttgtagcacgttgtagcactttttaggtgacacttttttgttgtagcac

SEQ ID NO: 50 - DNA, 48 bp, Artificial Sequence
  synthetic
gttgtagcactttttatttaggtgacactatagtttttgttgtagcac

SEQ ID NO: 51 - DNA, 50 bp, Artificial Sequence
  synthetic
gttgtagcacggtggcttttttcagctgatttttgttgtagcacggtggc

SEQ ID NO: 52 - DNA, 52 bp, Artificial Sequence
  synthetic
gttgtagcacggtggctttttatcagctgattttttgttgtagcacggtggc

SEQ ID NO: 53 - DNA, 54 bp, Artificial Sequence
  synthetic
gttgtagcacggtggctttttatatcgcgatattttttgttgtagcacggtggc

SEQ ID NO: 54 - DNA, 58 bp, Artificial Sequence
  synthetic
gttgtagcacggtggctttttatatgacgcgtcatattttttgttgtagcacggtggc

SEQ ID NO: 55 - DNA, 12 bp, Artificial Sequence
  synthetic
ttatgcgcataa

SEQ ID NO: 56 - DNA, 12 bp, Artificial Sequence
  synthetic
atatggccatat

SEQ ID NO: 57 - DNA, 12 bp, Artificial Sequence
  synthetic
ttaaggccttaa

SEQ ID NO: 58 - DNA, 12 bp, Artificial Sequence
  synthetic
aaaagcgctttt

SEQ ID NO: 59 - DNA, 12 bp, Artificial Sequence
  synthetic
taaagcgcttta

SEQ ID NO: 60 - DNA, 12 bp, Artificial Sequence
  synthetic
tataccggtata